(19) United States
(12) Patent Application Publication
(10) Pub. No.: US 2026/0364023 A1
(43) Pub. Date: Nov. 19, 2026

(54) LIPID COMPLEX NANOPARTICLE FOR LUNG-TARGETED DELIVERY OF NUCLEIC ACID THERAPEUTIC AGENT

(71) Applicant: IMMORNA (HANGZHOU) BIOTECHNOLOGY CO., LTD., Hangzhou (CN)

(72) Inventors: Neng Wang, Hangzhou (CN); Zhijun Guo, Hangzhou (CN); Zihao Wang, Hangzhou (CN); Hongyue Wu, Hangzhou (CN)

(21) Appl. No.: 19/630,431

(22) Filed: Mar. 27, 2026

Related U.S. Application Data
(63) Continuation of application No. PCT/CN2024/121624, filed on Sep. 27, 2024.

(30) Foreign Application Priority Data
Sep. 28, 2023 (WO) PCT/CN2023/122744

(51) Int. Cl. A61K 9/51; A61K 9/00; A61K 31/7105; A61K 31/711; A61K 47/18; A61K 47/24; A61K 47/34

(52) U.S. Cl. CPC A61K 9/5123; A61K 9/007; A61K 31/7105; A61K 31/711; A61K 47/186; A61K 47/24; A61K 47/34

(57) ABSTRACT

The present disclosure relates to the field of drug delivery system composing nanoparticles, in particular lipid nanoparticles (LNPs), and messenger RNA (mRNA), as well as methods for lung-targeted delivery of the nanoparticles.

### REFERENCE TO RELATED APPLICATION

This application is a continuation application filed under 35 U.S.C. 111 (a), based on International Patent Application No. PCT/CN2024/121624, filed on Sep. 27, 2024, which claims foreign priority to and the benefit of the filing date under 35 U.S.C. 365 (b) of International Patent Application No. PCT/CN2023/122744, filed on Sep. 28, 2023, the entire contents of each of the above-referenced applications, including all drawings and sequences are incorporated herein by reference.

### BACKGROUND OF THE INVENTION

In recent years, mRNA-based therapies have shown great therapeutic potential as vaccines or therapies, after the development of the past decades. For example, the U.S. Food and Drug Administration (FDA) recently approved two mRNA vaccines (mRNA-1273 and BNT162b2) against coronavirus disease 2019 (COVID-19), which vaccines have been hugely successful in preventing the spread of the COVID-19 epidemic.

However, the therapeutic potential of mRNA-based therapy has extended far beyond the field of vaccines, including therapies against infectious diseases, chronic diseases, rare genetic disorders, and tumor, all can be treated and/or prevented by designing specific mRNA encoding targeted proteins or antigens.

As is wildly known, mRNA can be easily degraded by the ubiquitous RNase existing everywhere in air or in vivo. Moreover, because mRNA molecules have large amounts of negative charges, they cannot be internalized by negatively charged cell membrane due to strong electrostatic repulsion. Furthermore, endosomal enzymes can also degrade mRNA. These factors limit the direct delivery of mRNA in vitro or in vivo. Thus, great efforts were devoted into the development of a variety of nanocarriers used for mRNA delivery in the past decades. Among these nanocarriers, LNP is the most widely used for its mature preparation process, high safety, outstanding protection effect, and excellent delivery efficiency.

Commonly, mRNA co-formulated with LNP predominantly accumulates in the liver and spleen following systemic delivery. Thus, much of the clinical interest involving LNPs is tightly related to hepatic diseases.

However, targeted mRNA delivery to extrahepatic organ or tissue still remains a major challenge, limiting the application scope of nucleic acid (siRNA or mRNA) therapies. Other therapies, such as small chemical molecules and proteins, also require an effective targeted delivery system to reduce adverse effects from system distribution of them. Thus, there is an urgent need for a targeted delivery system to address the current dilemma in precise targeted organ delivery.

Targeted therapy to the lungs has been studied in several genetic and refractory lung diseases, such as idiopathic pulmonary fibrosis (IPF). IPF is a progressive, fatal lung disease. Conventional chemotherapy is the main treatment for IPF. For example, nintedanib and pirfenidone are approved by the U.S. FDA to treat IPF. However, several undesirable side effects were observed in preclinical trials in many patients, such as critical nausea and diarrhea.

In recent years, gene therapy is increasingly considered as a potential alternative therapeutic option. However, administration of therapeutic agents via the respiratory route to IPF patients is still challenging, due to the decreased respiratory function, thickened epithelial cells in the airway and lung, and inevitably increased mucosal secretion in these patients. Thus, there is an urgent need for a delivery strategy that precisely delivers therapeutic agents to the lung after systemic administration.

To address these issues, several additional targeted delivery systems based on lipid have been developed, such as selective organ-targeting lipid nanoparticles (SORT LNP). SORT LNP dopes a high percentage of permanently cationic lipid, commonly DOTAP with 50% of total lipids, as the fifth component of lipid nanoparticle (LNP). However, SORT LNP has a large percentage (typically over 75%) of charged lipids, including ionizable and permanent cationic lipid, which may cause toxicity in clinical applications. Furthermore, tissue targeting is achieved with the concomitant loss of expression efficiency.

### SUMMARY OF THE INVENTION

The invention described herein provides a lipid-based targeted delivery system with excellent lung targeting. The delivery system described herein can be used to deliver any cargo molecule of interest, including (but not limited to polynucleotides or analogs/mimics thereof, polypeptides or mimics/analogs thereof, lipids, polysaccharides, and small molecules). For example, the polynucleotides that can be delivered using the delivery system described herein includes mRNA that can be selectively/preferentially delivered to lung tissues, while simultaneously maintaining high expression efficiency of the delivered mRNA.

The delivery system described herein is characterized, in part, by its overall low cationic lipid content, thus reducing the potential of toxicity.

More specifically, in one illustrative embodiment, the delivery system of the invention is a targeted lipid complex nanoparticle (tLCNP) comprising five or six types of lipids, including two different types of charged lipid such as ionizable lipid and permanently cationic lipid, one or two types of phospholipids, one or two types of sterols, and one type of stealth lipid derivatives such as PEGylated lipid. The lung-selective or lung-targeted delivery, and high expression efficiency of the therapeutic agent enables more handily regulation of the nucleic acid therapies the subject delivery system delivers, conferring this system a distinct advantage over the reported targeted delivery systems.

More detailed embodiments of the invention are provided herein in the numbered paragraphs below.
1. A targeted lipid complex nanoparticle (tLCNP) that preferentially delivers a therapeutic agent encompassed/formulated within the tLCNP to the lungs of a mammal, the tLCNP comprises:
   (1) a first lipid composition comprising an ionizable lipid C1 and a cationic lipid C2;
   (2) a second lipid composition comprising a first phospholipid PI and a second phospholipid PII;
   (3) a steroid; and,
   (4) a stealth lipid;
wherein:
(A) the ionizable lipid C1 has the formula of C1-a, C1-b, C1-c or C1-d:

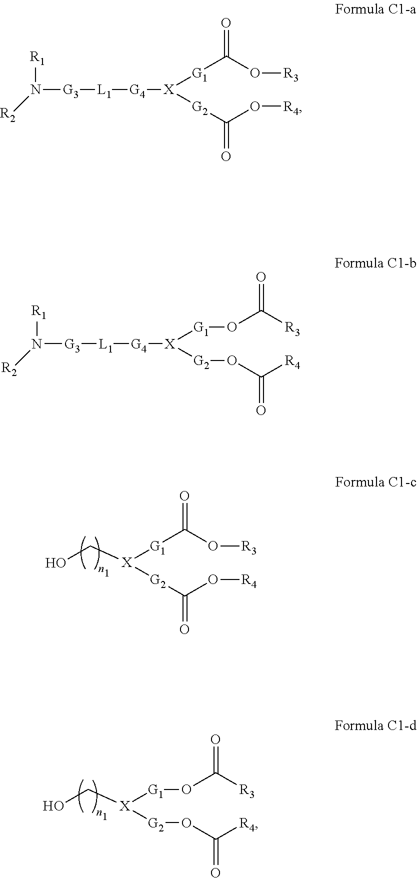

wherein:
   R₁ and R₂ are each independently —CH₃, —(CH₂)₁₋₈—CH₃, or —(CH₂)₁₋₈—OH;
   G₁ and G₂ are independently —(CH₂)₁₋₁₀—;
   G₃ and G₄ are independently —(CH₂)₀₋₅—;
   L₁ is —O—(C═O)—O—, —O—(C═O)—, —(C═O)—O—, —S—S—, —O—;
   R₃ and R₄ are each independently

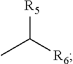

R₅ is —H, or —(CH₂)₇CH₃, —(CH═CH)(CH₂)₅CH₃, C1-14 alkyl, or C₁₋₁₄ alkenyl;
R₆ is —(CH₂)₇CH₃, —(CH═CH)(CH₂)₅CH₃, C₁₋₂₀ alkyl, or C₁₋₂₀ alkenyl;
X is C or

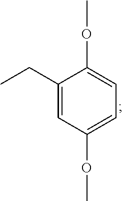

and,
n₁ is an integer of 2-8;
(B) the cationic lipid C2 has the formula of C2-a, C2-b, C2-c or C2-d:

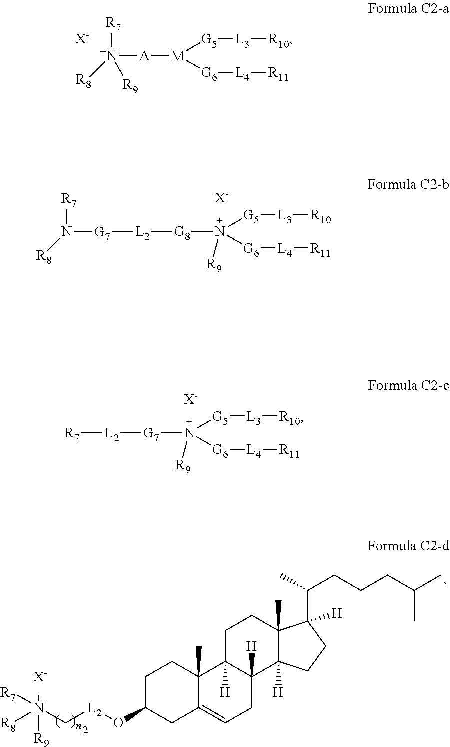

wherein:
   R₇, R₈, and R_{y} are each independently —CH₃, —(CH₂)₁₋₈—CH₃, or —(CH₂)₁₋₈—OH;
   R₁₀ and R₁₁ are each independently

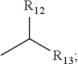

R₁₂ is —H, —(CH₂)₇CH₃, —(CH═CH)(CH₂)₅CH₃, C₁₋₁₄ alkyl, or C₁₋₁₄ alkenyl; R₁₃ is —(CH₂)₇CH₃, —(CH═CH)(CH₂)₅CH₃, C₁₋₂₀ alkyl, or C₁₋₂₀ alkenyl;
M is carbon or nitrogen;
A is —CH₂—, —G₇-L₂-G₈- or —(CH₂)₁₋₅—(C═O)—, wherein-(C═O)— is linked to M;
G₅, G₆, G₇ and G₈ are independently —(CH₂)₀₋₁₀—;
n₂ is an integer of 0-10;
L₂, L₃, and L₄ are each independently —CH₂—, —O—(C═O)—O—, —O—(C═O)—, —(C═O)—O—, —S—S—, —O—, —S—, —HN—(C═O)—;
X⁻ is monovalent anion (e.g., a halo group), such as Cl— and Br;
(C) optionally, the first phospholipid PI has the formula of PI:

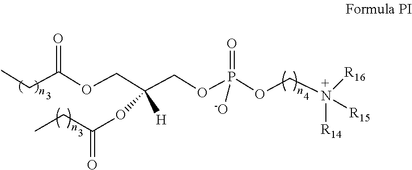

wherein R₁₄, R₁₅, and R₁₆ are each independently —H, —CH₃, —(CH₂)₁₋₆—CH₃, or —(CH₂)₁₋₆—OH;
n₃ in each instance is independently an integer of 5-20; and,
n₄ is an integer of 1-5; and,
(D) optionally, the second phospholipid PII has the formula of PII:

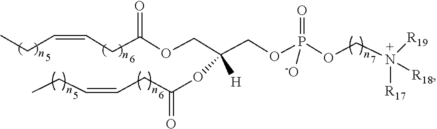

wherein R₁₇, R₁₈, and R₁₉ are each independently —H, —CH₃, —(CH₂)₁₋₆—CH₃, or —(CH₂)₁₋₆—OH;
n₅ and n₆, in each instance, is independently an integer of 5-8;
n₇ is an integer of 1-5.
2. The tLCNP of paragraph 1, wherein the steroid comprises, consists essentially of, or consists of cholesterol.
3. The tLCNP of paragraph 1 or 2, wherein the stealth lipid comprises, consists essentially of, or consists of a PEGylated-lipid having a PEG (—O—CH₂—CH₂—) repeat, such as DMG-PEG2000

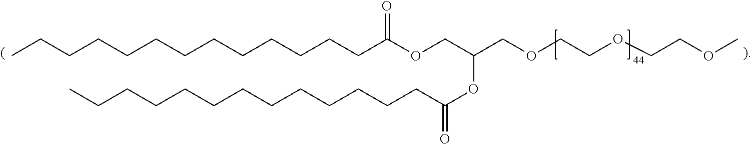

4. The tLCNP of paragraph 1, wherein R₅ comprises H, —(CH₂)₃CH₃, —(CH₂)₅CH₃, —(CH₂)₇CH₃, or —(CH₂)₉CH₃;
5. The tLCNP of paragraph 1, wherein R₅ comprises H, —(CH═CH)(CH₂)₃CH₃, —(CH₂)₃ (CH═CH) CH₂CH₃, —(CH═CH)(CH₂)₄CH₃, or —(CH═CH)(CH₂)₅CH₃, or —(CH═CH)(CH₂)₇CH₃;
6. The tLCNP of paragraph 1, wherein R₆ comprises-(CH₂)₃CH₃, —(CH₂)₅CH₃, —(CH₂)₇CH₃, or —(CH₂)₉CH₃;
7. The tLCNP of paragraph 1, wherein R₆ comprises-(CH═CH)(CH₂)₃CH₃, —(CH₂)₃ (CH═CH) CH₂CH₃, —(CH═CH)(CH₂)₄CH₃, —(CH═CH)(CH₂)₅CH₃, or —(CH═CH)(CH₂)₇CH₃;
8. The tLCNP of paragraph 1, wherein R₆ comprises H, —(CH₂)₃CH₃, —(CH₂)₅CH₃, —(CH₂)₇CH₃, —(CH₂)₉CH₃, or —(CH₂)₁₁CH₃;
9. The tLCNP of paragraph 1, wherein R₆ comprises H, —(CH═CH)(CH₂)₃CH₃, —(CH₂)₃ (CH═CH) CH₂CH₃, —(CH═CH)(CH₂)₄CH₃, —(CH═CH)(CH₂)₅CH₃, —(CH═CH)(CH₂)₇CH₃, or —(CH₂)₇ (CH═CH)(CH₂)₇CH₃;
10. The tLCNP of paragraph 1, wherein R₇ comprises-(CH₂)₃CH₃, —(CH₂)₅CH₃, —(CH₂)₇CH₃, —(CH₂)₉CH₃, or —(CH₂)₁₁CH₃;
11. The tLCNP of paragraph 1, wherein R₇ comprises-(CH═CH)(CH₂)₃CH₃, —(CH₂)₃ (CH═CH) CH₂CH₃, —(CH═CH)(CH₂)₄CH₃, or —(CH═CH)(CH₂)₅CH₃, —(CH═CH)(CH₂)₇CH₃, or —(CH₂)₇ (CH═CH)(CH₂)₇CH₃;
12. The tLCNP of any one of paragraphs 1-11, wherein said ionizable lipid C1 comprises

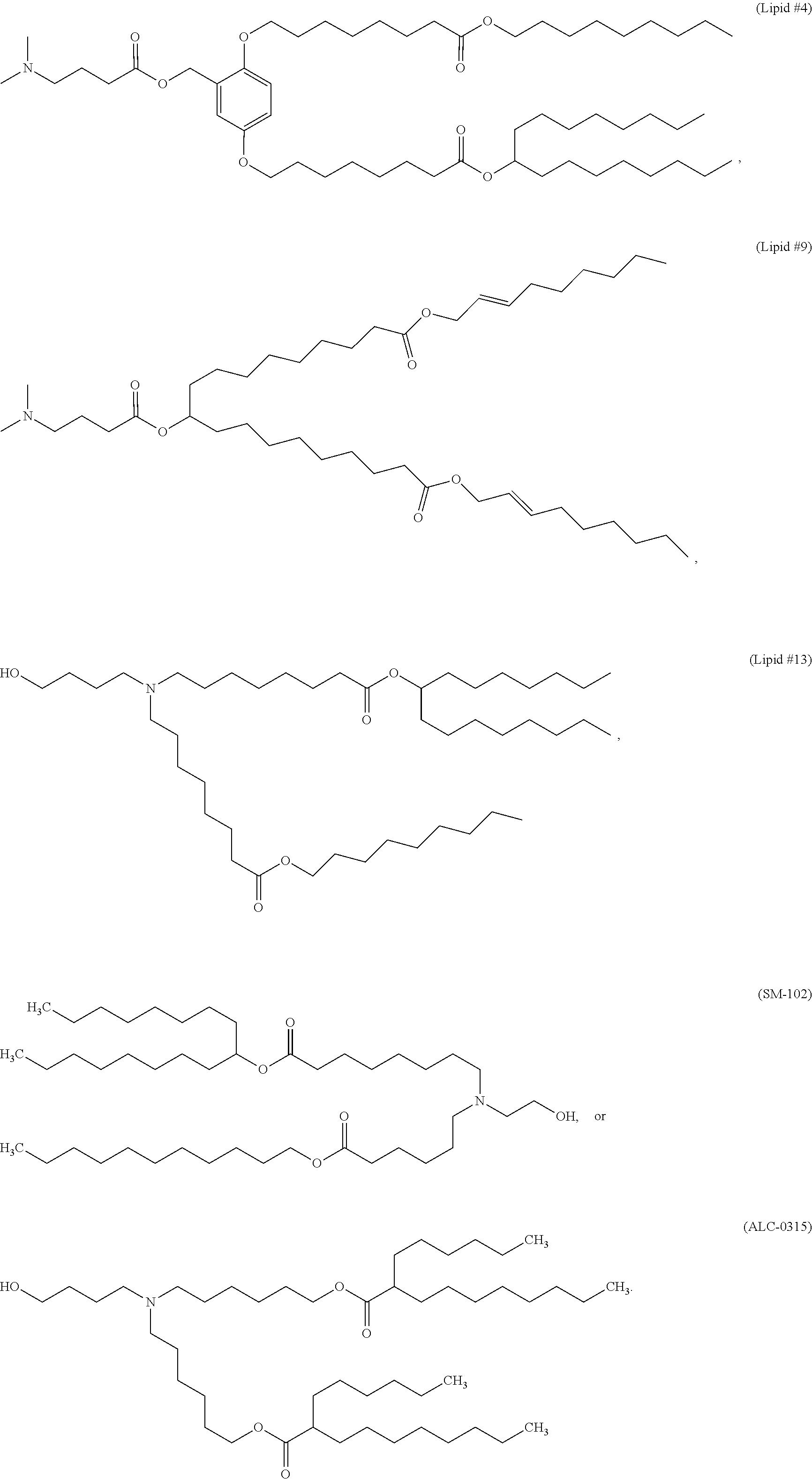

13. The tLCNP of any one of paragraphs 1-12, wherein said cationic lipid C2 comprises

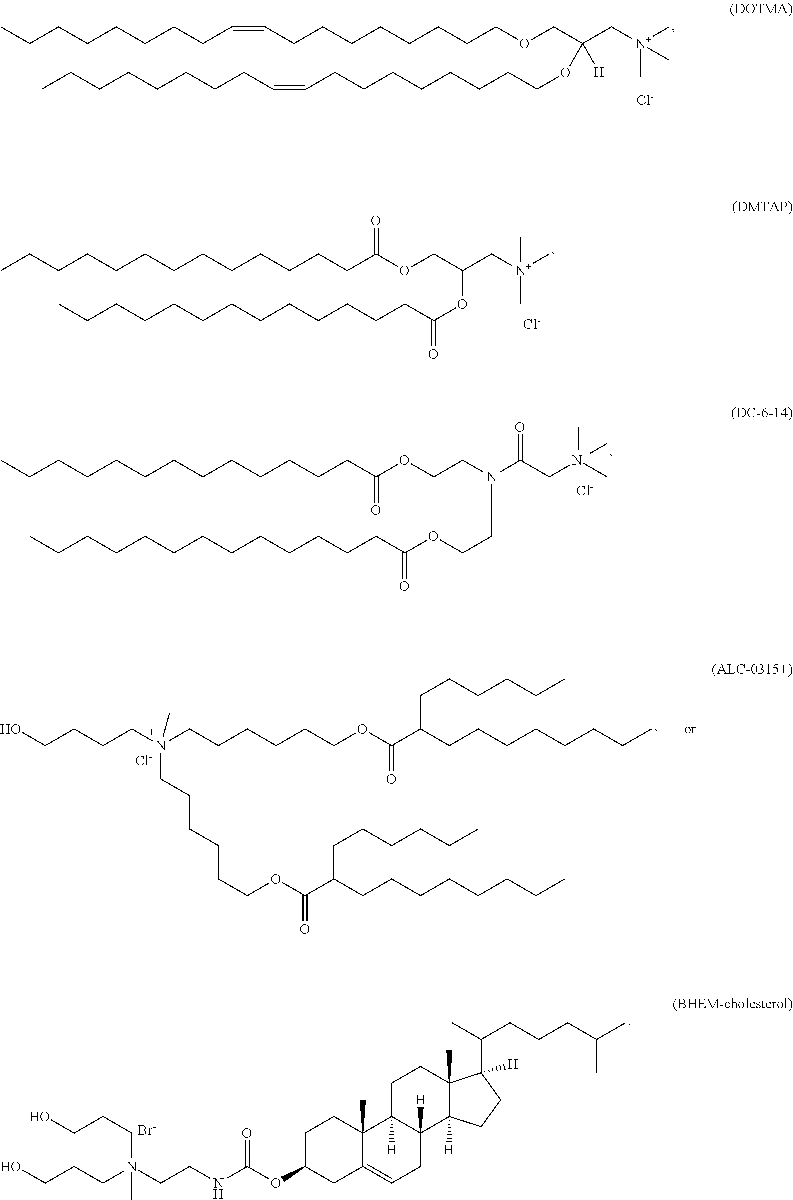

14. The tLCNP of any one of paragraphs 1-13, wherein said first phospholipid PI comprises

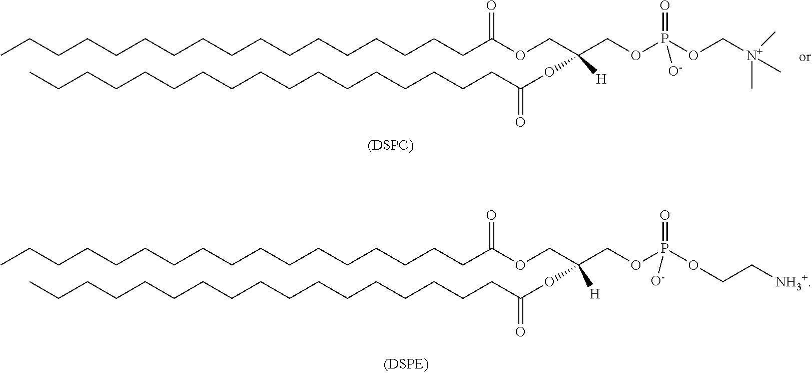

15. The tLCNP of any one of paragraphs 1-14, wherein said second phospholipid PII comprises

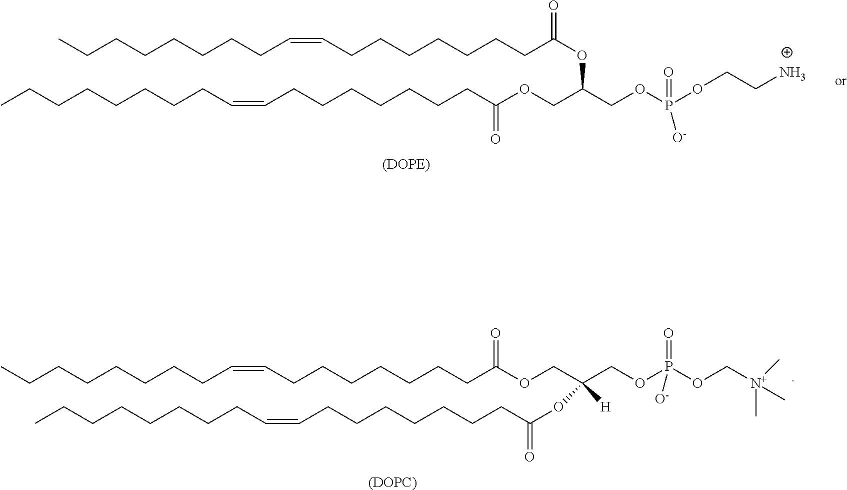 [SMILES: CCCCCCCC/C=C\CCCCCCCC(=O)OC[C@H](COP(=O)([O-])OCC[NH3+])OC(=O)CCCCCCC/C=C\CCCCCCCC ; CCCCCCCC/C=C\CCCCCCCC(=O)OC[C@H](COP(=O)([O-])OC[N+](C)(C)C)OC(=O)CCCCCCC/C=C\CCCCCCCC ; "(DOPC)"]

16. The tLCNP of any one of paragraphs 1-15, wherein the molar ratio of the cationic lipid C2 over the ionizable lipid C1 is about 1:3 to about 4:1, about 1:1 to about 4:1, about 2:1 to about 4:1, about 3:1 to about 4:1, about 1:1, about 2:1, about 3:1, or about 4:1.
17. The tLCNP of paragraph 16, wherein the cationic lipid C2 is DOTMA, and the ionizable lipid C1 is Lipid #4.
18. The tLCNP of any one of paragraphs 1-17, wherein the molar percentage of (1) (or the first lipid composition) over (1)-(4) is about 30-70%, about 40-60%, about 50-60%, about 30%, about 40%, about 50%, about 60%, or about 70%.
19. The tLCNP of paragraph 18, wherein the molar ratio of the cationic lipid C2 (such as DOTMA) over the ionizable lipid C1 (such as Lipid #4) is about 2:1.
20. The tLCNP of any one of paragraphs 1-19, comprising:
   (a) about 30%-70%, about 40%-60%, or about 50% of the first lipid composition in molar percentage;
   (b) about 5%-30% (such as about 5-15%, or about 10%) of the second lipid composition in molar percentage, optionally, the molar ratio of the first phospholipid PI (such as DSPC) over the second phospholipid PII (such as DOPC) is from 1:0 (100% DSPC) to 1:2 (33% DSPC), such as 2:1 (67% DSPC) or 1:1 (50% DSPC);
   (c) about 10%-40% (such as about 35-40%, or about 38.5%) of the steroid (such as cholesterol) in molar percentage;
   (d) about 0%-5% (such as about 2-3%, or about 1.5%) of the stealth lipid (such as DMG-PEG2000) in molar percentage; or
   (e) a combination of any of the above, wherein the total molar percentage of the combination is 100%
21. The tLCNP of any one of paragraphs 1-20, wherein the therapeutic agent comprises a polynucleotide (including DNA and RNA, such as mRNA, siRNA, miRNA, ASO, and pDNA), a hydrophobic and/or hydrophilic small molecule compound (e.g., with a molecular weight of under 1000 Da, 750 Da, 500 Da, 250 Da, or 100 Da), or a bioactive lipid (such as fatty acid, or monoglyceride).
22. The tLCNP of paragraph 21, wherein the therapeutic agent comprises a polynucleotide, such as an RNA (e.g., an siRNA, a miRNA, a pri-miRNA, a messenger RNA (mRNA), a clustered regularly interspaced short palindromic repeats (CRISPR) related nucleic acid, a single guide RNA (sgRNA), a CRISPR-RNA (crRNA), a trans-activating crRNA (tracrRNA), a plasmid DNA (pDNA), a transfer RNA (tRNA), an antisense oligonucleotide (ASO), a guide RNA, a double stranded DNA (dsDNA), a single stranded DNA (ssDNA), a single stranded RNA (ssRNA), and a double stranded RNA (dsRNA)).
23. The tLCNP of paragraph 22, wherein the RNA is a self-replicating mRNA, a non-self-replicating mRNA, or a circular RNA.
24. The tLCNP of paragraph 22 or 23, wherein the N/P molar ratio (first lipid composition (permanent cationic lipid+ionizable lipid) lipid Nitrogen: polynucleotide Phosphate) of the tLCNP is about 4, 6, or 8, such as 6.
25. The tLCNP of any one of paragraphs 1-24, wherein at least about 60%, 70%, 75%, 80%, 85%, 90%, or 95% of the therapeutic agent is delivered to the lungs when the tLCNP is administered to the mammal.
26. The tLCNP of any one of paragraphs 1-25, wherein no more than about 30%, 25%, 20%, 15%, 10%, or 5% of the therapeutic agent is delivered to the liver (or other non-lung tissues combined) when the tLCNP is administered to the mammal.
27. The tLCNP of any one of paragraphs 1-26, which on average has a size less than 200 nm, less than 150 nm, less than 100 nm, less than 90 nm, or less than 80 nm.
28. The tLCNP of any one of paragraphs 1-27, which has a polydispersity index (PDI) of between about 0.10-0.25, or between about 0.15-0.20.
29. The tLCNP of any one of paragraphs 1-28, wherein about 1-15%, about 2-10%, or about 3-5% of the therapeutic agent is delivered to the spleen when the tLCNP is administered to the mammal.
30. A pharmaceutical composition comprising the tLCNP of any one of paragraphs 1-29; and a pharmaceutically acceptable carrier or excipient.
31. A method of delivering a therapeutic agent selectively or preferentially to the lungs of a mammal, comprising administering to the mammal the therapeutic agent encompassed or formulated in the tLCNP of any one of paragraphs 1-29 to the mammal.
32. The method of paragraph 31, wherein the mammal is a human.
33. The method of paragraph 31 or 32, wherein the tLCNP is injected into the mammal intravenously.
34. The method of any one of paragraphs 31-33, wherein the therapeutic agent comprises a polynucleotide, such as an RNA.
35. The method of any one of paragraphs 31-34, wherein the mammal has a lung disease, and the therapeutic agent is effective to treat the lung disease.
36. The method of paragraph 35, wherein the lung disease is lung cancer, asthma, pneumothorax, atelectasis, bronchitis (including chronic bronchitis), bronchiolitis, bronchiectasis, COPD, emphysema, lung infection (e.g., pneumonia, COVID-19, bacterial infection, viral infection), cystic fibrosis, idiopathic pulmonary fibrosis (IPF), pulmonary edema, pulmonary embolus, lymphangioleiomyomatosis (LAM), or pulmonary hypertension.
37. A method of preparing the tLCNP of any one of paragraphs 1-29, the method comprising mixing a first organic liquid phase comprising the first lipid composition, the second lipid composition, the steroid, and the stealth lipid of any one of paragraphs 1-29, in an organic solvent (e.g., ethanol) that is miscible with water; and a second aqueous phase comprising the therapeutic agent of any one of paragraphs 1-29, in order to form the tLCNP.
38. The method of paragraph 37, further comprising step-wisely purification of the tLCNP by a Tangential Flow Filtration (TFF) system.
39. The method of paragraph 37 or 38, wherein said organic solvent in the first organic liquid phase comprises, consists essentially of, or consists of ethanol.
40. The method of any one of paragraphs 37-39, wherein the first organic liquid phase and the second aqueous phase are mixed in a microfluidic device, optionally with a volume ratio of 1:3, and/or optionally at a total flow rate of about 10-15 mL/min. (e.g., about 12 mL/min.).
41. The method of any one of paragraphs 37-40, wherein the aqueous phase comprises a 50 mM citrate buffer (pH 5.0).
42. The method of any one of paragraphs 38-41, wherein the step-wisely purification is performed in the TFF system in a citrate buffer (pH 5.0) and Tris-HCl buffer (pH 7.2) containing 7.5% sucrose.

It should be understood that any one embodiment described herein, including embodiments only described in the examples or claims, can be combined with any one or more other embodiments of the disclosure, unless such combination is improper, or is expressly disclaimed.

### BRIEF DESCRIPTION OF THE DRAWINGS

FIG. 1 is a schematic drawing (not to scale) illustrating the composition and component ratios of an embodiment of the lung-targeting (tLCNP) of the invention.

FIG. 2 shows the structural formulae of several representative molecules used to produce the subject lung-targeting (tLCNP).

FIGS. 3-6 show that tLCNP prepared with different Lipid #4/DOTMA ratio allow mRNA to be systematically delivered from liver to spleen and then to lung. (FIG. 3) In vivo and ex vivo imaging of mice intravenously injected luciferase mRNA@tLCNP with different Lipid #4/DOTMA ratio at 6 hours post-injection. (FIG. 4) Quantification data of the organs expressing luciferase (Data are shown as mean±SD, n=3 biologically independent animals). (FIG. 5) Heat map of luciferase expression in different organ of mice treated by above samples. (FIG. 6) Relative luciferase expression in each organ demonstrated that lung targeted expression could be readily enhanced by regulating Lipid #4/DOTMA ratio.

FIGS. 7-10 show tLCNP prepared with different ionizable lipids, including MC3, Lipid #4, Lipid #13, and Lipid #9. (FIG. 7) In vivo and ex vivo imaging of mice intravenously injected luciferase mRNA@tLCNP with different ionizable lipids at 6 hours post-injection. (FIG. 8) Quantification data of the organs expressing luciferase (Data are shown as mean±SD, n=3 biologically independent animals). (FIG. 9) Heat map of luciferase expression in different organ of mice treat by above samples. (FIG. 10) Relative luciferase expression in each organ showed that tLCNP prepared with other XH series ionizable lipids (including Lipid #13 and Lipid #9) also readily achieved lung-targeted expression, whereas MC3 did not.

FIGS. 11-14 show tLCNP prepared with different percentage of total charged lipids: Lipid #4+DOTMA. (FIG. 11) In vivo and ex vivo imaging of mice intravenously injected luciferase mRNA@tLCNP with different percentage of total Lipid #4+DOTMA at 6 hours post-injection. (FIG. 12) Quantification data of the organs expressing luciferase (Data are shown as mean±SD, n=3 biologically independent animals). (FIG. 13) Heat map of luciferase expression in different organ of mice treat by above samples. (FIG. 14) Relative luciferase expression in each organ demonstrated that lung targeting efficiency could be readily elevated by increase total cationic lipid percentage, whether too high ratio leads to the comprising of expression intensity.

FIGS. 15-18 show tLCNP prepared with different N/P ratio of 4, 6, and 8. (FIG. 15) In vivo and ex vivo imaging of mice intravenously injected luciferase mRNA@tLCNP with different N/P ratio of 4, 6, and 8, at 6 hours post-injection. (FIG. 16) Quantification data of the organs expressing luciferase (Data are shown as mean±SD, n=3 biologically independent animals). (FIG. 17) Heat map of luciferase expression in different organ of mice treat by above samples. (FIG. 18) Relative luciferase expression in each organ demonstrated that the N/P ratio of 6 is the optimal for lung-targeting.

FIGS. 19-22 show tLCNP prepared with different DSPC/DOPE ratio. (FIG. 19) In vivo and ex vivo imaging of mice intravenously injected luciferase mRNA@tLCNP with different DSPC/DOPE ratio at 6 hours post-injection. (FIG. 20) Quantification data of the organs expressing luciferase (Data are shown as mean±SD, n=3 biologically independent animals). (FIG. 21) Heat map of luciferase expression in different organ of mice treat by above samples. (FIG. 22) Relative luciferase expression in each organ demonstrated that lung targeted expression could be readily enhanced by regulating phospholipid composition and ratio.

FIGS. 23-26 show tLCNP prepared with different DOPC/DSPC ratio or DOPC/DOPE ratio. (FIG. 23) In vivo and ex vivo imaging of mice intravenously injected luciferase mRNA@tLCNP with different DOPC/DSPC ratio or DOPC/DOPE ratio at 6 hours post-injection. (FIG. 24) Quantification data of the organs expressing luciferase (Data are shown as mean±SD, n=3 biologically independent animals). (FIG. 25) Heat map of luciferase expression in different organ of mice treat by above samples. (FIG. 26) Relative luciferase expression in each organ demonstrated that lung targeted expression could be readily enhanced by regulating phospholipid composition and ratio.

FIGS. 27-30 show comparison of SORT LNP and LNP:DOTMA with our innovated tLCNP regarding to the lung-targeting. (FIG. 27) In vivo and ex vivo imaging of mice intravenously injected luciferase mRNA loaded SORT LNP, LNP:DOTMA, and tLCNP at 6 hours post-injection. (FIG. 28) Quantification data of the organs expressing luciferase (Data are shown as mean±SD. (n=3 biologically independent animals). (FIG. 29) Heat map of luciferase expression in different organ of mice treat by above samples. (FIG. 30) Relative luciferase expression in each organ demonstrated that tLCNP show a significant targeting efficiency and expression intensity over SORT LNP or LNP (DOTMA).

FIGS. 31-33 show lung-targeting evaluation of tLCNP on rabbit. (FIG. 31) Ex vivo imaging of rabbit intravenously injected luciferase mRNA loaded tLCNP at 6 hours post-injection. (FIG. 32) Quantification data of the organs expressing luciferase. (FIG. 33) Elisa test determining RBD concentration on liver, lung, and spleen of rabbits administrated tLCNP (RBD) and PBS control 4 hours post-injection.

FIG. 34 show the daily mortality rate of mice that were injected with PBS (control) or with IL12-CBD mRNA@tLCNP. Autopsy was performed to determine tumor burden in the lungs, liver, spleen, kidney, and GI. Statistical significance was achieved by Log-rank (Mantel-Cox) test.

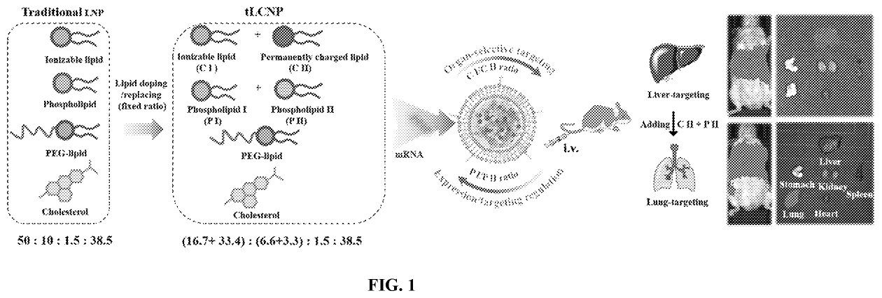

### DETAILED DESCRIPTION OF THE INVENTION

The invention described herein provides a lipid nanoparticle (e.g., tLCNP) formulation that preferentially delivers a cargo molecule or composition encompassed/encapsulated/or otherwise formulated in the LNP, such as a polynucleotide or analogs/mimics thereof (e.g., DNA, RNA, morpholino, PNA (peptide nucleic acid), XNA (Xeno Nucleic Acid), locked nucleic acid (LNA), glycol nucleic acid (GNA), threose nucleic acid (TNA), and hexitol nucleic acids (HNA)), proteins or polypeptides, small molecule compounds (such as those with a molecular weight of no more than 1,500 Da, 1,000 Da, 800 Da, 500 Da, 300 Da, 250 Da, 200 Da, 150 Da etc.), and the like.

The invention described herein is partly based on the surprising discovery that certain ionizable lipids (such as the CI ionizable lipids described herein), when mixed with certain permanently charged lipids (such as the CII permanently charged lipids described herein), can dramatically shift the tissue distribution of tLCNPs prepared with such lipid mixtures, from predominantly to liver, to partially liver partially spleen, to predominantly to lung. This allows tissue-specific delivery of the cargo molecules carried by the LNP particles, such as lung-preferential or lung-specific delivery of the cargo molecules, if not lung-exclusive delivery of the cargo molecules.

The invention described herein is also partly based on the surprising discovery that certain phospholipid components of the tLCNPs can be fine-tuned to boost targeting and/or expression efficiency of the polynucleotide cargo carried by the tLCNPs.

Thus one aspect of the invention described herein provides a targeted lipid complex nanoparticles (tLCNP) that preferentially delivers a cargo molecule (e.g., a therapeutic agent) encompassed/formulated within the tLCNP to the lungs of a mammal, the tLCNP comprises:
(1) a first lipid composition comprising an ionizable lipid C1 and a cationic lipid C2; (2) a second lipid composition comprising a first phospholipid PI and a second phospholipid PII; (3) a steroid; and, (4) a stealth lipid; wherein:
   (A) the ionizable lipid C1 has the formula of C₁-a, C₁-b, C₁-c or C₁-d:

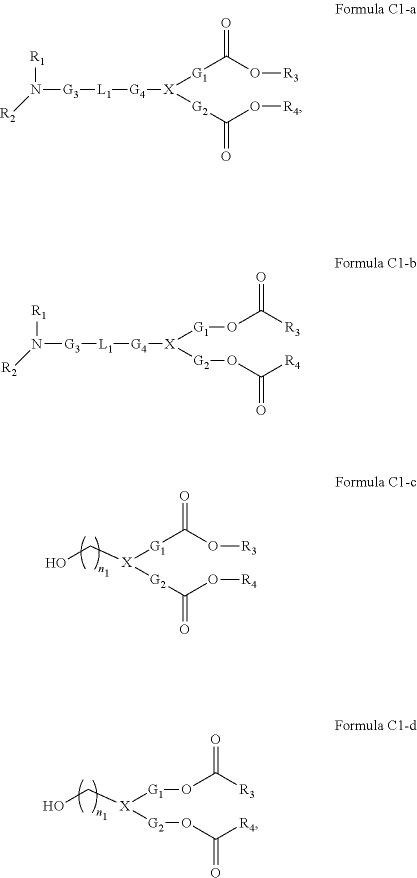

wherein:
   R₁ and R₂ are each independently —CH₃, —(CH₂)₁₋₈—CH₃, or —(CH₂)₁₋₈—OH; Gi and G₂ are independently —(CH₂)₁₋₁₀—;
   G₃ and G₄ are independently —(CH₂)₀₋₅—;
   L₁ is —O—(C═O)—O—, —O—(C═O)—, —(C═O)—O—, —S—S—, —O—;
   R₃ and R₄ are each independently

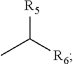

R₅ is —H, —(CH₂)₇CH₃, —(CH═CH)(CH₂)₅CH₃, C₁₋₁₄ alkyl, or C₁₋₁₄ alkenyl;
R₆ is —(CH₂)₇CH₃, —(CH═CH)(CH₂)₅CH₃, C₁₋₂₀ alkyl, or C₁₋₂₀ alkenyl;
X is C or

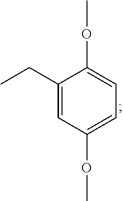

and,
n₁ is an integer of 2-8;
(B) the cationic lipid C2 has the formula of C₂-a, C₂-b, C₂-c or C₂-d:

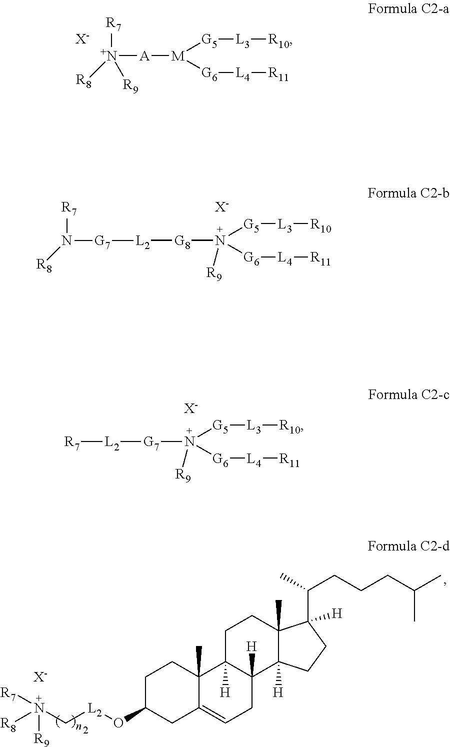

wherein:
   R₇, R₈, and R₉ are each independently —CH₃, —(CH₂)₁₋₈—CH₃, or —(CH₂)₁₋₈—OH;
   R₁₀ and R₁₁ are each independently

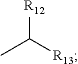

R₁₂ is —H, —(CH₂)₇CH₃, —(CH═CH)(CH₂)₅CH₃, C₁₋₁₄ alkyl, or C₁₋₁₄ alkenyl;
R₁₃ is —(CH₂)₇CH₃, —(CH═CH)(CH₂)₅CH₃, C₁₋₂₀ alkyl, or C₁₋₂₀ alkenyl;
M is carbon or nitrogen;
A is —CH₂—, —G₇-L₂-G₈- or —(CH₂)₁₋₅—(C═O)—, wherein-(C═O)— is linked to M;
G₅, G₆, G₇ and G₈ are independently —(CH₂)₀₋₁₀—;
n₂ is an integer of 0-10;
L₂, L₃, and L₄ are each independently —CH₂—, —O—(C═O)—O—, —O—(C═O)—, —(C═O)—O—, —S—S—, —O—, —S—, —HN—(C═O)—;
X⁻ is monovalent anion (e.g., a halo group), such as Cl— and Br;
(C) optionally, the first phospholipid PI has the formula of PI:

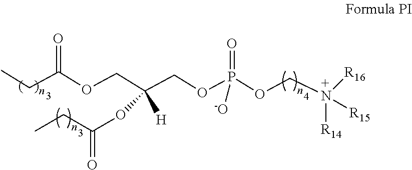

wherein R₁₄, R₁₅, and R₁₆ are each independently —H, —CH₃, —(CH₂)₁₋₆—CH₃, or —(CH₂)₁₋₆—OH;
n₃ in each instance is independently an integer of 5-20; and,
n₄ is an integer of 1-5; and,
(D) optionally, the second phospholipid PII has the formula of PII:

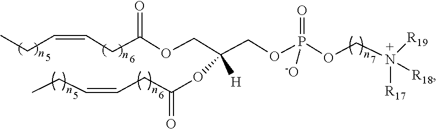

wherein R₁₇, R₁₈, and R₁₉ are each independently —H, —CH₃, —(CH₂)₁₋₆—CH₃, or —(CH₂)₁₋₆—OH;
n₅ and n₆, in each instance, is independently an integer of 5-8;
n₇ is an integer of 1-5.

While not wishing to be bound by any particular theory, the ionizable lipids described herein circumvent certain undesirable cytotoxicity associated with cationic lipids. The ionizable lipids described herein has a transient cationic charge that is acquired at low pH (e.g., about pH 5-6, such as about pH5.0 or 5.5), forming reverse micelles that encapsulate the cargo molecules (such as polynucleotides) in the LNP core. These lipids have near-neutral charge at physiological pH, and deliver the cargo molecules with much reduced toxicity or without cytotoxicity.

The ionizable lipids useful for the invention described herein can be prepared based on, for example, WO2022166213A1, WO2023104114A2 (incorporated herein by reference).

In certain embodiments, the C₁₋₁₄ alkyl or C₁₋₂₀ alkyl is methyl, ethyl, propyl, butyl, pentyl, hexyl, heptyl, octyl, nonyl, decyl, undecyl, dodecyl, tridecyl, tetradecyl, or a branched variant thereof (such as isopropyl, 3-pentyl, sec-pentyl etc.).

In certain embodiments, the C₁₋₁₄ alkenyl or C₁₋₂₀ alkenyl is ethylene, propylene, 1-butene, 2-butene, isobutylene, 1-pentene, 2-pentene, 2-methyl-1-butene, 3-methyl-1-butene, 2-methyl-2-butene, 1-hexene, 2-hexene, 3-hexene, 2-methyl-1-pentene, 3-methyl-1-pentene, 4-methyl-1-pentene, 2-methyl-2-pentene, 3-methyl-2-pentene, 4-methyl-2-pentene, 2,3-dimethyl-1-butene, 3,3-dimethyl-1-butene, 2,3-dimethyl-2-butene, 2-ethyl-1-butene, etc. In certain embodiments, the alkenyl group contains 1, 2, 3, 4, or 5 double bonds.

In certain embodiments, the phospholipid comprises phosphatidylcholine, such as distearoyl phosphatidylcholine (DSPC), 1,2-Dioleoyl-sn-glycero-3-phosphocholine (DOPC), and/or phosphoethanolamine, such as 1,2-distearoyl-sn-glycero-3-phospho-ethanolamine (DSPE), 1,2-dioleoyl-sn-glycero-3-phosphoethanolamine (DOPE).

In certain embodiments, the first phospholipid PI in the tLCNP has the formula of PI, as defined above. In certain embodiments, the second phospholipid PII in the tLCNP has the formula of PII, as defined above. In certain embodiments, the first phospholipid PI in the tLCNP has the formula of PI, as defined above, and the second phospholipid PII in the tLCNP has the formula of PII, as defined above.

In certain embodiments, the steroid comprises, consists essentially of, or consists of a sterol lipid, such as cholesterol.

While not wishing to be bound by any particular theory, sterol lipids, such as cholesterol, fill lipid membrane packing defects and provide structural integrity to the subject tLCNP. They may also aid in membrane fusion of the tLCNP and target cell.

In certain embodiments, the sterol lipid comprises a steroid and/or a secosteroid, such as zoosterols, phytosterols and mycosterols and combinations and mixtures thereof. In some embodiments, the sterol lipid comprises a cholesterol and/or phytosterol, and/or in particular at least one secosterol, preferably at least a calciferol (vitamin D); in particular where the steroid and/or secosteroid, in particular the sterol, has a Griffin HLB value of at most 1.5 and/or in particular where the steroid and/or secosteroid, in particular the sterol, has a log P value of at least 5.

In certain embodiments, the sterol lipid is cholesterol, cholesterol sulfate, or a derivative thereof, such as 7α-hydroxy cholesterol (which may improve the delivery of an oligonucleotide cargo).

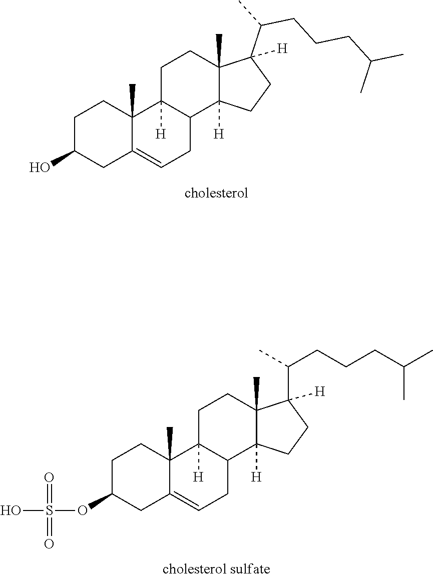

Again, while not wishing to be bound by any particular theory, PEGylated lipids in the tLCNP described herein prevent serum protein adsorption, and inhibits uptake by the mononuclear phagocyte system (MPS), which a major obstacle in the delivery of LNPs. In certain embodiments, the PEGylated lipids also contain terminal functional groups, such as amine or maleimide, which can be used to conjugate other molecules that improve cellular targeting and uptake.

In certain embodiments, the PEGylated lipid/stealth lipid comprises, consists essentially of, or consists of a PEGylated-lipid having a PEG (—O—CH₂—CH₂—) repeat, such as DMG-PEG2000

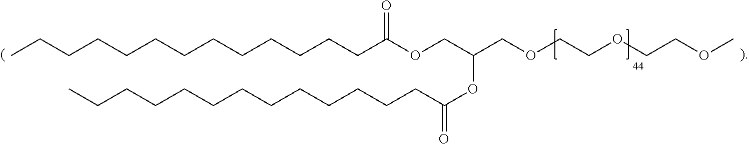

In certain embodiments, the PEGylated lipid/stealth lipid comprises, consists essentially of, or consists of C-DMG-PEG (2000)/PEG (2000)-C-DMG or ALC-0159.

In certain embodiments, the ionizable lipid C1 comprises

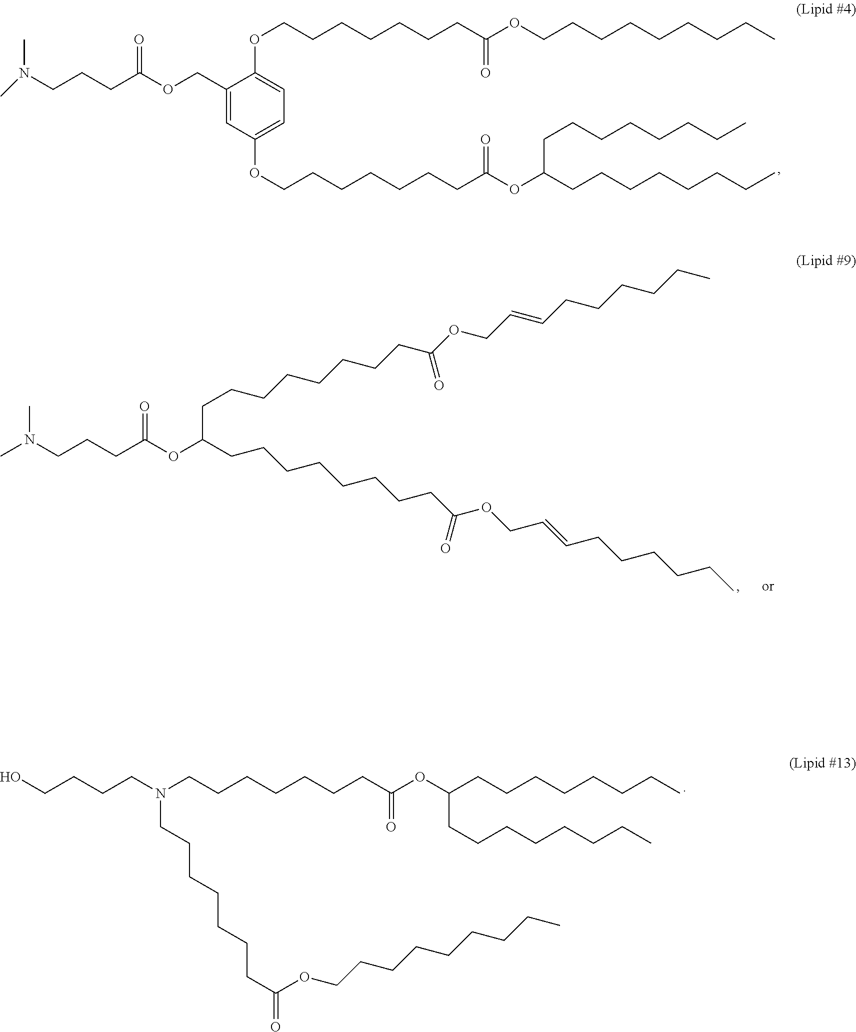

In certain embodiments, the ionizable lipid C1 may also be replaced by lipids such as ALC-0315 (shown below) or SM-102 (shown below)

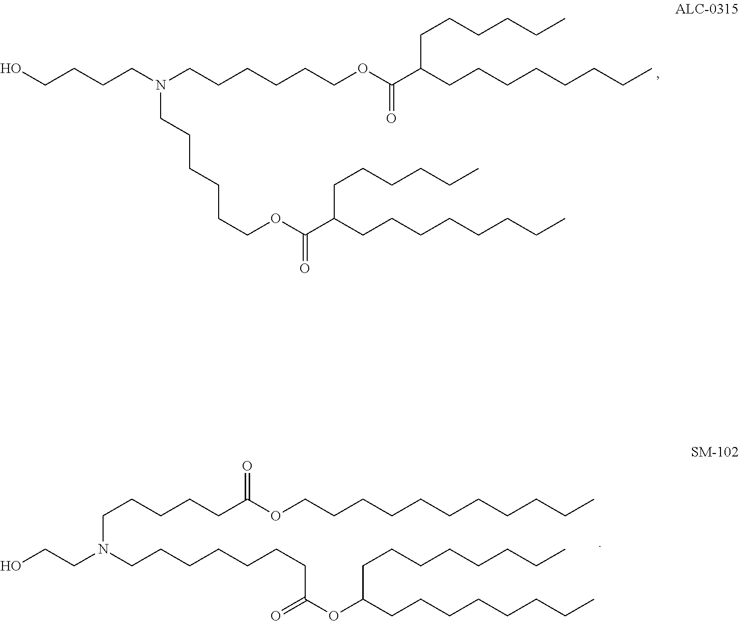 [SMILES: CCCCCCCCC(CCCCCC)C(=O)OCCCCCCN(CCCCO)CCCCCCOC(=O)C(CCCCCC)CCCCCCCC ; CCCCCCCCCCCOC(=O)CCCCCN(CCO)CCCCCCCC(=O)OC(CCCCCCCC)CCCCCCCC]

In certain embodiments, the cationic lipid C2 comprises

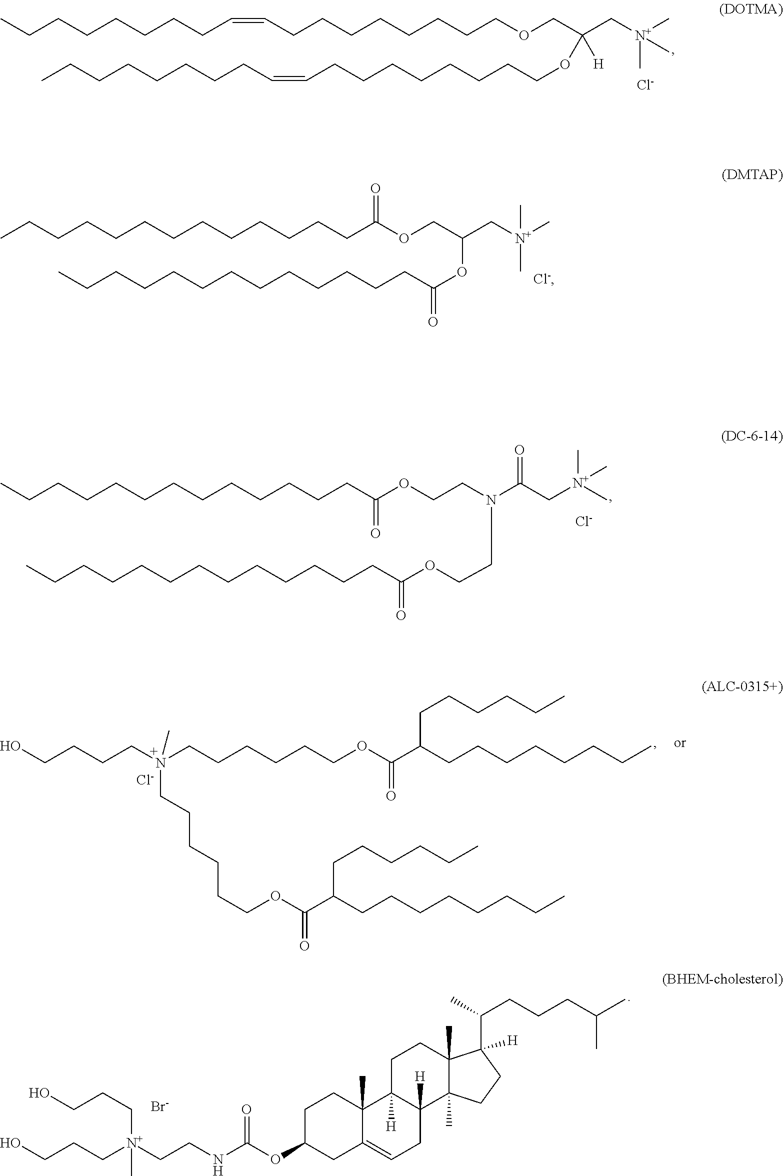

Glycerophospholipids are a class of phospholipid that contains a hydrophilic head group and two hydrophobic fatty acyl tails attached to a glycerol backbone. The hydrophilic head determines the charge of the tLCNP particle, which in theory can be neutral, anionic (negative), or cationic (positive). In certain embodiments, the phospholipid in the tLCNP is an anionic lipid, such as a hosphatidylglycerol (PG), a phosphatidylinositol (PI), a phosphatidylserine (PS), and a phosphatidic acid (PA).

In certain embodiments, the first phospholipid PI comprises

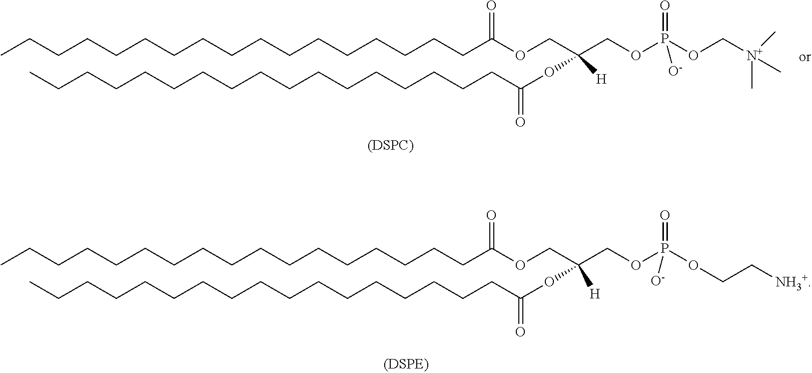

In certain embodiments, the second phospholipid PII comprises

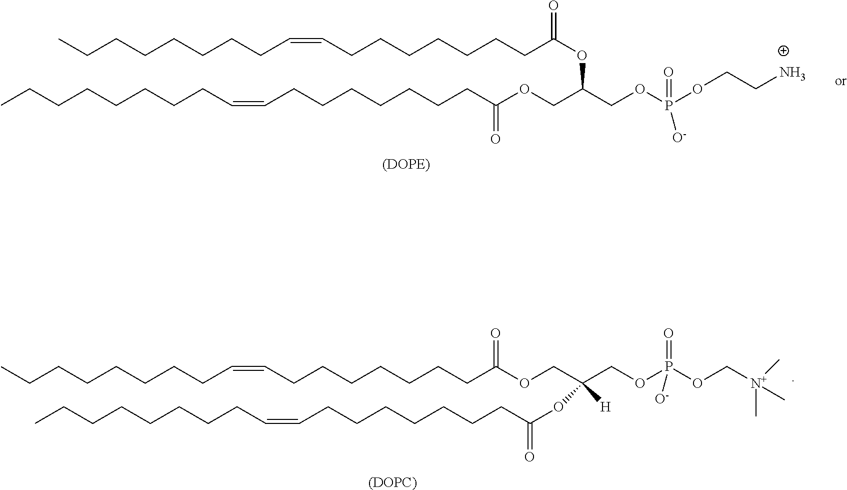 [SMILES: CCCCCCCC/C=C\CCCCCCCC(=O)OC[C@H](COP(=O)([O-])OCC[NH3+])OC(=O)CCCCCCC/C=C\CCCCCCCC ; CCCCCCCC/C=C\CCCCCCCC(=O)OC[C@H](COP(=O)([O-])OC[N+](C)(C)C)OC(=O)CCCCCCC/C=C\CCCCCCCC ; "(DOPC)"]

In certain embodiments, the molar ratio of the cationic lipid C2 over the ionizable lipid C1 is about 1:3 to about 4:1, about 1:1 to about 4:1, about 2:1 to about 4:1, about 3:1 to about 4:1, about 1:1, about 2:1, about 3:1, or about 4:1.

In certain embodiments, the cationic lipid C2 is DOTMA, and the ionizable lipid C1 is Lipid #4.

In certain embodiments, the molar percentage of (1) (or the first lipid composition) over (1)-(4) is about 30-70%, about 40-60%, about 50-60%, about 30%, about 40%, about 50%, about 60%, or about 70%.

In certain embodiments, the molar ratio of the cationic lipid C2 (such as DOTMA) over the ionizable lipid C1 (such as Lipid #4) is about 2:1.

In certain embodiments, the tLCNP described herein comprises: (a) about 30%-70%, about 40%-60%, or about 50% of the first lipid composition in molar percentage; (b) about 5%-30% (such as about 5-15%, or about 10%) of the second lipid composition in molar percentage, optionally, the molar ratio of the first phospholipid PI (such as DSPC) over the second phospholipid PII (such as DOPC) is from 1:0 (100% DSPC) to 1:2 (33% DSPC), such as 2:1 (67% DSPC) or 1:1 (50% DSPC); (c) about 10%-40% (such as about 35-40%, or about 38.5%) of the steroid (such as cholesterol) in molar percentage; (d) about 0%-5% (such as about 2-3%, or about 1.5%) of the stealth lipid (such as DMG-PEG2000) in molar percentage; or the combination of any of the above, wherein the total molar percentage of the combination is 100%.

In certain embodiments, the tLCNP comprises an internal aqueous phase that solubilizes hydrophilic cargo molecules, such as oligonucleotides in the tLCNPs.

In certain embodiments, the cargo is a therapeutic agent comprising a polynucleotide (including DNA and RNA, such as mRNA, siRNA, miRNA, ASO, and pDNA), a hydrophobic and/or hydrophilic small molecule compound (e.g., with a molecular weight of under 1000 Da, 750 Da, 500 Da, 250 Da, or 100 Da), or a bioactive lipid (such as fatty acid, or monoglyceride).

In certain embodiments, the therapeutic agent comprises a polynucleotide, such as an RNA (e.g., an siRNA, a miRNA, a pri-miRNA, a messenger RNA (mRNA), a clustered regularly interspaced short palindromic repeats (CRISPR) related nucleic acid, a single guide RNA (sgRNA), a CRISPR-RNA (crRNA), a trans-activating crRNA (tracrRNA), a plasmid DNA (pDNA), a transfer RNA (tRNA), an antisense oligonucleotide (ASO), a guide RNA, a double stranded DNA (dsDNA), a single stranded DNA (ssDNA), a single stranded RNA (ssRNA), and a double stranded RNA (dsRNA)).

In certain embodiments, the RNA is a self-replicating mRNA, a non-self-replicating mRNA, or a circular RNA.

In certain embodiments, the cargo is a small molecule. In certain embodiments, the small molecule is a hydrophilic drug solubilized in the aqueous compartments of tLCNP. In certain embodiments, the small molecule comprises Doxorubicin, an antitumor antibiotic, is a hydrophilic drug.

In certain embodiments, the cargo is a bioactive lioid. Fatty acids, monoglycerides, and other bioactive lipids may also be incorporated into the subject tLCNP system of the invention. In certain embodiments, the bioactive lipid comprises lauric acid, linoleic acid, oleic acid, C₆ ceramide, or sphingolipid.

In certain embodiments, the N/P molar ratio (first lipid composition (permanent cationic lipid+ionizable lipid) lipid Nitrogen: polynucleotide Phosphate) of the tLCNP is about 4, 6, or 8, such as 6.

As used herein, the “N/P molar ratio” refers to the ratio of the charged nitrogen (N) molar amount in the first lipid composition (permanent cationic lipid+ionizable lipid): the phosphorus (P) molar amount of the polynucleotide in the LNP formulation.

In certain embodiments, at least about 60%, 70%, 75%, 80%, 85%, 90%, or 95% of the therapeutic agent is delivered to the lungs when the tLCNP is administered to the mammal.

In certain embodiments, no more than about 30%, 25%, 20%, 15%, 10%, or 5% of the therapeutic agent is delivered to the liver (or other non-lung tissues combined) when the tLCNP is administered to the mammal.

In certain embodiments, the tLCNP described herein has on average a size less than about 200 nm, less than about 150 nm, less than about 100 nm, less than about 90 nm, or less than about 80 nm.

In certain embodiments, the tLCNP described herein has a polydispersity index (PDI) of between about 0.10-0.25, or between about 0.15-0.20.

In certain embodiments, about 1-15%, about 2-10%, or about 3-5% of the therapeutic agent is delivered to the spleen when the tLCNP is administered to the mammal.

Another aspect of the invention provides a pharmaceutical composition/formulation comprising the tLCNP described herein; and a pharmaceutically acceptable carrier or excipient.

In certain embodiments, the pharmaceutical composition/formulation is formulated for systemic administration, e.g. intravenous injection/administration.

The phrase “systemic administration” as used herein refers to its meaning as is generally accepted in the art. The term generally refers to methods or techniques of administering a molecule, drug, agent or compound in a manner resulting in in vivo systemic absorption or accumulation of drugs in the blood stream followed by distribution throughout the entire body. Systemic administration includes in ovo administration.

In certain embodiments, systemic administration include pulmonary (inhalation, nebulization etc.), intravenous, subcutaneous, catheterization, nasopharyngeal, or oral/gastrointestinal administration as is generally known in the art.

Further non-limiting examples of systemic administration methods of the invention include buccal, sublingual, parenteral (i.e., intravenously, intraperitoneally, subcutaneously, or intramuscularly), local rectal administration or other local administration resulting in absorption or accumulation of the administered composition in the blood stream followed by distribution throughout the entire body.

In one embodiment, systemic administration includes insufflation and inhalation.

In one embodiment, systemic administration includes intravenous or intraperitoneal administration by a bolus injection (see, e.g., U.S. Pat. No. 5,286,634). In another embodiment, the tLCNPs are administered subcutaneously. In a further embodiment, the tLCNPs and pharmaceutical compositions thereof are administered intravenously (IV).

Intravenously administered tLCNPs described herein preferentially if not exclusively directs/delivers the cargo molecules to the lung. However, certain tLCNP formulations described herein also delivers significant amount of the cargo molecules to the spleen, and may be used for spleen-targeted delivery.

The pharmaceutical composition/formulation can be prepared by any method known or hereafter developed in the art of pharmaceutics. In general, such preparatory methods include the step of bringing the active ingredient (e.g., the tLCNP formulations described herein) into association with one or more excipients and/or one or more other accessory ingredients, and then, if necessary and/or desirable, shaping and/or packaging the product into a desired single- or multi-dose unit.

In certain embodiments a pharmaceutical composition may be prepared, packaged, and/or sold in bulk, as a single unit dose, and/or as a plurality of single unit doses.

As used herein, a “unit dose” is a discrete amount of the pharmaceutical composition comprising a predetermined amount of the therapeutic agent (e.g., mRNA, ASO, siRNA, small chemical drug molecule, etc.) encompassed/formulated within the tLCNP formulations described herein. The amount of the therapeutic agent (or an interger multiple thereof, such as 2×, 3×, 4× etc.,) is generally equal to the dosage of the therapeutic agent which would be administered to a subject.

In certain embodiments, a unit dose of the pharmaceutical composition may be further split into fractions thereof, such as ½, ⅓, ¼, ⅕ of a unit dose etc. Such fractions of the unit dose may be useful for certain patient populations such as pediatric patients.

The relative or exact amounts of therapeutic agent encompassed/formulated within tLCNP formulations described herein, the pharmaceutically acceptable excipient(s), and/or any additional ingredients in a pharmaceutical composition of the invention will vary, depending upon the identity, size, and/or condition of the subject treated and further depending upon the route by which the composition is to be administered.

In certain embodiments, the pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21.sup.st Edition, A. R. Gennaro, (Lippincott, Williams & Wilkins, Baltimore, Md., 2006) discloses various excipients used in formulating pharmaceutical compositions and known techniques for the preparation thereof. Except insofar as any conventional excipient is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, the pharmaceutically acceptable excipient is at least 95%, 96%, 97%, 98%, 99%, or 100% pure. In some embodiments, the excipient is approved for use in humans and veterinary use. In some embodiments, the excipient is approved by United States Food and Drug Administration. In some embodiments, the excipient is pharmaceutical grade. In some embodiments, the excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in the pharmaceutical formulations comprising the immunogenic nanoparticles described herein. Excipients such as cocoa butter and suppository waxes, coloring agents, coating agents, sweetening, flavoring, and perfuming agents can be present in the composition, according to the judgment of the formulator.

Illustrative diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Illustrative granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water-insoluble starch, calcium carboxymethyl cellulose, magnesium aluminum silicate (Veegum), sodium lauryl sulfate, quaternary ammonium compounds, etc., and combinations thereof.

Illustrative surface active agents and/or emulsifiers include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and Veegum [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN20®], polyoxyethylene sorbitan [TWEEN60®], polyoxyethylene sorbitan monooleate [TWEEN80®], sorbitan monopalmitate [SPAN40®], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ®45], polyoxyethylene hydrogenated castor oil, poly ethoxylated castor oil, polyoxymethylene stearate, and Solutol), sucrose fatty acid esters, polyethylene glycol fatty acid esters (e.g., CREMOPHOR®), polyoxyethylene ethers, (e.g., polyoxyethylene lauryl ether [BRIJ®30]), polyvinylpyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof.

Illustrative disintegrating agents include, but are not limited to, agar, calcium carbonate, potato or tapioca starch, alginic acid, certain silicates, and sodium carbonate.

Illustrative binding agents include, but are not limited to, starch (e.g., cornstarch and starch paste); gelatin; sugars (e.g., sucrose, glucose, dextrose, dextrin, molasses, lactose, lactitol, mannitol,); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan); alginates; polyethylene oxide; polyethylene glycol; inorganic calcium salts; silicic acid; polymethacrylates; waxes; water; alcohol; etc. and combinations thereof.

Illustrative preservatives may include antioxidants, chelating agents, antimicrobial preservatives, antifungal preservatives, alcohol preservatives, acidic preservatives, and other preservatives. Illustrative antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite. Illustrative chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and trisodium edetate. Illustrative antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal. Illustrative antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and sorbic acid. Illustrative alcohol preservatives include, but are not limited to, ethanol, polyethylene glycol, phenol, phenolic compounds, bisphenol, chlorobutanol, hydroxybenzoate, and phenylethyl alcohol. Illustrative acidic preservatives include, but are not limited to, vitamin A, vitamin C, vitamin E, beta-carotene, citric acid, acetic acid, dehydroacetic acid, ascorbic acid, sorbic acid, and phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, Germall 115, Germaben II, Neolone. TM., Kathon. TM., and EUXYL®. In certain embodiments, the preservative is an antioxidant. In other embodiments, the preservative is a chelating agent.

Illustrative buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Illustrative lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Illustrative oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Illustrative oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Liquid dosage forms for oral and parenteral administration include, but are not limited to, pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, therapeutic agent of the invention are mixed with solubilizing agents such as CREMOPHOR®, alcohols, oils, modified oils, glycols, polysorbates, cyclodextrins, polymers, and combinations thereof.

Injectable formulations, for example, sterile injectable aqueous or oleaginous suspensions may be formulated according to the known art using suitable dispersing or wetting agents and suspending agents. A sterile injectable preparation may be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable media prior to use.

Solid dosage forms for oral administration include capsules, tablets, pills, powders, and granules. In such solid dosage forms, the active ingredient is mixed with at least one inert, pharmaceutically acceptable excipient such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polethylene glycols and the like.

In certain embodiments the tLCNP-encapsulated therapeutic agentcan be in micro-encapsulated form with one or more excipients as noted above. Solid dosage forms of tablets, draggers, capsules, pills, and granules can be prepared with coatings and shells such as enteric coatings, release controlling coatings and other coatings well known in the pharmaceutical formulating art. In such solid dosage forms, active ingredient may be admixed with at least one inert diluent such as sucrose, lactose or starch. Such dosage forms may comprise, as is normal practice, additional substances other than inert diluents, e.g., tableting lubricants and other tableting aids such a magnesium stearate and microcrystalline cellulose. In the case of capsules, tablets and pills, dosage forms may comprise buffering agents. They may optionally comprise opacifying agents and can be of a composition that they release the therapeutic agent only, or preferentially, in a certain part of the intestinal tract, optionally, in a delayed manner. Examples of embedding compositions which can be used include polymeric substances and waxes.

In certain embodiments, dosage forms for topical and/or transdermal administration of therapeutic agent may include ointments, pastes, creams, lotions, gels, powders, solutions, sprays, inhalants and/or patches. Generally, active ingredient is admixed under sterile conditions with a pharmaceutically acceptable excipient and/or any needed preservatives and/or buffers as may be required. Additionally, the use of transdermal patches is also contemplated. Such patches often have the added advantage of providing controlled delivery of an active ingredient to the body. Such dosage forms may be prepared, for example, by dissolving and/or dispensing the active ingredient in the proper medium. Alternatively, or additionally, the rate may be controlled by either providing a rate controlling membrane and/or by dispersing the active ingredient in a polymer matrix and/or gel.

Suitable devices for use in delivering intradermal pharmaceutical compositions described herein include soluble microneedle array patches, e.g., as described in U.S. Patent Publication Nos: US 2019/0358441 A1, US 2019/0240469 A1, US 2019/0151638 A1, US 2019/0151638 A1, US 2018/0001070 A1, US 2017/0209553 A1, US 2016/0263362 A1, US 2017/0196966 A1, US 2016/0213908 A1, US 2016/0015952 A1, US 2015/0112250 A1, US 2012/0150023, and the like. Also contemplated are short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices that limit the effective penetration length of a needle into the skin, such as those described in PCT publication WO 99/34850 and functional equivalents thereof. Jet injection devices that deliver vaccines to the dermis via a liquid jet injector and/or via a needle that pierces the stratum comeum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/037705 and WO 97/013537, and the like. Ballistic powder/particle delivery devices that use compressed gas to accelerate vaccine in powder form through the outer layers of the skin to the dermis are suitable. Alternatively or additionally, conventional syringes may be used in the classical mantoux method of intradermal administration.

In certain embodiments formulations suitable for topical administration include, but are not limited to, liquid and/or semi liquid preparations such as liniments, lotions, oil in water and/or water in oil emulsions such as creams, ointments and/or pastes, and/or solutions and/or suspensions. In certain embodiments, topically-administrable formulations may, for example, comprise from about 1% to about 10% (w/w) therapeutic agent (immunogenic nanoparticles described herein), although the concentration of the active ingredient may be as high as the solubility limit of the active ingredient in the solvent. In certain embodiments, formulations for topical administration may further comprise one or more of the additional ingredients described herein.

In certain embodiments, a pharmaceutical composition comprising the tLCNP formulations described herein may be prepared, packaged, and/or sold in a formulation suitable for pulmonary administration via the buccal cavity. Such a formulation may, for example, comprise particles that comprise or contain the tLCNP-encapsulated therapeutic agent described herein. Such compositions are conveniently in the form of dry powders for administration using a device comprising a dry powder reservoir to which a stream of propellant may be directed to disperse the powder and/or using a self-propelling solvent/powder dispensing container such as a device comprising the active ingredient dissolved and/or suspended in a low-boiling propellant in a sealed container.

Low boiling propellants generally include liquid propellants having a boiling point of below 65 °F at atmospheric pressure. In certain embodiments the propellant may constitute 50% to 99.9% (w/w) of the composition, and the therapeutic agent may constitute 0.1% to 20% (w/w) of the composition. The propellant may further comprise additional ingredients such as a liquid non-ionic and/or solid anionic surfactant and/or a solid diluent (which may have a particle size of the same order as particles comprising the active ingredient).

In certain embodiments, pharmaceutical compositions comprising the therapeutic agent described herein formulated for pulmonary delivery may provide the nanoparticles in the form of droplets of a solution and/or suspension. In certain embodiments, such formulations may be prepared, packaged, and/or sold as aqueous and/or dilute alcoholic solutions and/or suspensions, optionally sterile, comprising the active ingredient, and may conveniently be administered using any nebulization and/or atomization device. In certain embodiments, such formulations may further comprise one or more additional ingredients including, but not limited to, a flavoring agent such as saccharin sodium, a volatile oil, a buffering agent, a surface active agent, and/or a preservative such as methylhydroxybenzoate. In certain embodiments, the droplets provided by this route of administration may have an average diameter in the range from about 0.1 μm up to about 200 μm.

In certain embodiments, the formulations described herein as being useful for pulmonary delivery are useful for intranasal delivery of a pharmaceutical composition of the invention. Another illustrative formulation suitable for intranasal administration is a coarse powder comprising the active ingredient and having an average particle from about 0.2 μm to about 500 pm. Such a formulation can be administered in the manner in which snuff is taken, i.e., by rapid inhalation through the nasal passage from a container of the powder held close to the nares.

In certain embodiments, formulations suitable for nasal administration may, for example, comprise from about as little as 0.1% (w/w) and as much as 100% (w/w) of the tLCNP formulations described herein, and may comprise one or more of the additional ingredients described herein. In certain embodiments, a pharmaceutical composition of the tLCNP formulations described herein may be prepared, packaged, and/or sold in a formulation suitable for buccal administration. Such formulations may, for example, be in the form of tablets and/or lozenges made using conventional methods, and may, for example, 0.1% to 20% (w/w) of the tLCNP formulations described herein, the balance comprising an orally dissolvable and/or degradable composition and, optionally, one or more of the additional ingredients described herein. Alternately, in certain embodiments, formulations suitable for buccal administration may comprise a powder and/or an aerosolized and/or atomized solution and/or suspension comprising the immunogenic nanoparticles. In certain embodiments, such powdered, aerosolized, and/or aerosolized formulations, when dispersed, may have an average particle and/or droplet size in the range from about 0.1 μm to about 200 μm, and may further comprise one or more of the additional ingredients described herein.

General considerations in the formulation and/or manufacture of pharmaceutical agents may be found, for example, in Remington: The Science and Practice of Pharmacy 21 st ed., Lippincott Williams & Wilkins, 2005.

In some embodiments, a therapeutically effective amount of the tLCNP formulations described herein is delivered to a patient and/or animal prior to, simultaneously with, and/or after diagnosis with a disease. In some embodiments, a therapeutic amount of an inventive composition is delivered to a patient and/or animal prior to, simultaneously with, and/or after onset of symptoms the disease. In some embodiments, the amount of tLCNP formulations described herein is sufficient to treat, alleviate, ameliorate, relieve, delay onset of, inhibit progression of, reduce severity of, and/or reduce incidence of one or more symptoms or features of the disease. In some embodiments, the amount of the tLCNP formulations described herein is sufficient to elicit a detectable immune response in a subject. In some embodiments, the amount of tLCNP formulations described herein is sufficient to elicit a detectable T cell response in a subject. In some embodiments, the amount of the tLCNP formulations described herein is sufficient to elicit a detectable antibody and T cell response in a subject. In some embodiments, an advantage of the nanoparticles provided is that the nanoparticles can elicit potent responses with a much lower concentration of antigen than required with a conventional control.

The compositions, according to the methods described herein, can be administered using any amount and any route of administration effective for treatment. The exact amount required will vary from subject to subject, depending on the species, age, and general condition of the subject, the severity of the infection, the particular composition, its mode of administration, its mode of activity, and the like. The compositions comprising the tLCNP formulations described herein described herein are typically formulated in dosage unit form for ease of administration and uniformity of dosage. It will be understood, however, that the total daily usage of the compositions will be decided by the attending physician within the scope of sound medical judgment. The specific therapeutically effective dose (or prophylactically effective dose) level for any particular subject or organism will depend upon a variety of factors including the disorder being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

In some embodiments, the pharmaceutical compositions described are administered by a variety of routes, including oral, intravenous, intramuscular, intra-arterial, intramedullary, intrathecal, subcutaneous, intraventricular, transdermal, intradermal, rectal, intravaginal, intraperitoneal, topical (as by powders, ointments, creams, and/or drops), mucosal, nasal, buccal, enteral, sublingual; by intratracheal instillation, bronchial instillation, and/or inhalation; and/or as an oral spray, nasal spray, and/or aerosol. Specifically, contemplated routes are oral administration, intravenous injection, intramuscular injection, and/or subcutaneous injection. In some embodiments, the nanoparticles are administered parenterally. In some embodiments, the nanoparticles are administered intravenously. In some embodiments, the nanoparticles are administered orally.

In general the most appropriate route of administration will depend upon a variety of factors including the nature of the tLCNP formulations described herein (e.g., its stability in the environment of the gastrointestinal tract), the condition of the subject (e.g., whether the subject is able to tolerate oral administration), etc.

In certain embodiments, the therapeutic agent described herein may be administered in amounts ranging from about 0.1 μg/kg to about 100 μg/kg, from about 0.2 μg/kg to about 50 μg/kg, from about 0.3 μg/kg to about 25 μg/kg, from about 0.4 μg/kg to about 10 μg/kg, from about 0.5 μg/kg to about 2.5 μg/kg, of subject body weight per day, one or more times a day, to obtain the desired therapeutic effect. In certain embodiments, the desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In certain embodiments, the desired dosage may be delivered 2 weeks apart, one-month apart, two-month apart, three-month apart, four-month apart, five-month apart, or six-month apart.

In some embodiments, the present invention encompasses “therapeutic cocktails” comprising populations of the tLCNP formulations described herein. In some embodiments, all of the tLCNP formulations described herein within a population of nanoparticles comprise a single species of cargo. In some embodiments, different nanoparticles within a population of nanoparticles comprise different cargos.

Another aspect of the invention provides a method of delivering a therapeutic agent selectively or preferentially to the lungs of a mammal, comprising administering to the mammal the therapeutic agent encompassed or formulated in the tLCNP described herein.

In certain embodiments, the mammal is a human. In certain embodiments, the mammal is a non-human animal, a non-human mammal, or a non-human primate (e.g., a livestock animal such as a cow, ox or bull, sheep, goat, horse, pig, camel, etc; a pet such as a cat or a dog; or a rodent such as rabbit, mouse, rat, hamster, Guinea Pig, etc.).

In certain embodiments, the tLCNP is injected into the mammal intravenously.

In certain embodiments, the therapeutic agent comprises a polynucleotide, such as an RNA (e.g., mRNA).

In certain embodiments, the mammal has a lung disease, and the therapeutic agent is effective to treat the lung disease.

In certain embodiments, the lung disease is lung cancer, asthma, pneumothorax, atelectasis, bronchitis (including chronic bronchitis), bronchiolitis, bronchiectasis, COPD, emphysema, lung infection (e.g., pneumonia, COVID-19, bacterial infection, viral infection), cystic fibrosis, idiopathic pulmonary fibrosis (IPF), pulmonary edema, pulmonary embolus, lymphangioleiomyomatosis (LAM), or pulmonary hypertension.

Another aspect of the invention provides a method of preparing the tLCNP of the invention, the method comprising mixing a first organic liquid phase comprising the first lipid composition, the second lipid composition, the steroid, and the stealth lipid of the disclosure described herein, in an organic solvent (e.g., ethanol) that is miscible with water; and a second aqueous phase comprising the therapeutic agent of the disclosure, in order to form the tLCNP.

In certain embodiments, the method is carried out in a microfluidic mixing device/chip. Automated microfluidic devices or microfluidic chips are fast and efficient methods to prepare LNPs. These devices enable rapid mixing in a highly controllable, reproducible manner that achieves homogeneous LNPs and high encapsulation efficiency. In these devices, individual streams of the ethanolic lipid mixture and aqueous oligonucleotide solution are rapidly combined. The subject tLCNPs form as the two streams mix and are collected into a single collection tube. Parameters such as the flow rate ratio and total flow rate can be altered to fine-tune tLCNPs.

In certain embodiments, the method is carried out in a T- or Y-junction mixer. These mixers can be assembled with common and inexpensive laboratory materials. A T- or Y-connecter can be fitted with two inlets coupled to individual syringes containing the lipid mixture or oligonucleotide solution and one outlet to direct the LNPs into a collection tube. Optionally, the inlet flow rates can be controlled with syringe pumps.

In certain embodiments, the method is carried out using ethanol-injection. Mixing of the ethanolic lipid mixture and aqueous oligonucleotide solution is performed with the aid of a magnetic stir plate. Inject the ethanolic lipid mixture into the aqueous acidic oligonucleotide solution with constant stirring and continue to mix for 30 minutes.

A simpler alternative method to ethanol-injection is hand mixing. Transfer the ethanolic lipid mixture into the aqueous acidic oligonucleotide solution and mix for 15 seconds by rapid pipetting. Leave the mixture undisturbed for 10 minutes.

In certain embodiments, the method further comprises extrusion to reduce particle size and generate uniform particle size distributions.

In certain embodiments, the method further comprises dialyzing the tLCNPs in storage buffer using appropriate molecular weight cut-off (MWCO) tubing. This step removes unencapsulated cargo, excess lipid components, and ethanol from the final preparation. Dialysis also adjusts the pH of the LNPs from the acidic preparation buffer to the neutral storage solution. This step is generally performed with ethanol-injection or hand-mixing methods.

In certain embodiments, the method further comprises filtration to sterilize LNPs. In certain embodiments, the filtration step is carried out by using a 0.22 μm filter to remove bacteria or other contaminants.

In certain embodiments, the method further comprises step-wisely purification of the tLCNP by a Tangential Flow Filtration (TFF) system.

In certain embodiments, the organic solvent in the first organic liquid phase comprises, consists essentially of, or consists of ethanol.

In certain embodiments, the first organic liquid phase and the second aqueous phase are mixed in a microfluidic device, optionally with a volume ratio of 1:3, and/or optionally at a total flow rate of about 10-15 mL/min. (e.g., about 12 mL/min.).

In certain embodiments, the aqueous phase comprises a 50 mM citrate buffer (pH 5.0).

In certain embodiments, the step-wisely purification is performed in the TFF system in a citrate buffer (pH 5.0) and Tris-HCl buffer (pH 7.2) containing 7.5% sucrose.

In certain embodiments, the method further comprises measuring the size (e.g., average size and/or size distribution) of tLCNPs. In certain embodiments, the size is determined by Dynamic light scattering (DLS) measurements, using, for example, a Zetasizer Nano ZS from Malvern Instruments Ltd.

In certain embodiments, the method further comprises measuring encapsulation efficiency (EE %) of the cargo, such as mRNA. In certain embodiments, the EE % is determined by using, for example, RIBOGREEN® assay. RIBOGREEN® is a dye that exhibits fluorescence when bound to single stranded mRNA but cannot enter the LNPs.

In certain embodiments, the method further comprises imaging a sample of the tLCNP by cryogenic electron microscopy (Cryo-EM) to investigate its nano-precipitated core structure. The cryo-EM images can demonstrate the encapsulation of mRNA in what appears to be a tLCNP, exhibiting a multilayered core structure.

With the general aspect of the invention described herein, the following examples further illustrate specific aspects of the invention, which are not limiting in any respect. Any specific embodiments described in the examples are also specific embodiments of the invention, and can be/intended to be combined with other aspects of the invention or other embodiments described herein above.

### Examples

### Example 1 Manufacture of Self-Replicating mRNA and Traditional mRNA

In Vitro Transcription of srRNA

Self-replicating mRNA (srRNA) was produced from a backbone plasmid vector based on a Trinidad donkey Venezuelan equine encephalitis strain (VEEV) alphavirus genome according the reported method (Blakney et al., ACS Nano 13:5920-5930, 2019, incorporated herein by reference).

Then, post-transcriptionally capped srRNA was synthesized as previously described (Mckay et al., Nat. Commun. 11, 3523., 2020, incorporated herein by reference). Specifically, uncapped RNA was pre-prepared using 1 μg linearized DNA template in a MEGAScript reaction (Ambion, UK) as manufacturer's protocol described. Transcripts were then purified by LiCl precipitation overnight at −20 °C, pelleted by centrifugation at 14,000 rpm for 20 min at 4 °C, washed once with 70% ethanol, centrifuged at 14,000 rpm for 5 min at 4 °C and then resuspended in UltraPure H₂O. After that, purified transcripts were capped through the ScriptCap m7G capping system (CellScript, Madison, WI, USA) and ScriptCap 2′-O-methyltransferase kit (CellScript) simultaneously as the manufacturer's protocol described. Capped transcripts were then purified by LiCl precipitation with the same procedure as detailed above, resuspended in UltraPure H₂O and stored at −80 °C for following use.

In Vitro Transcription of mRNA

The manufacturing of traditional luciferase mRNA consists of three steps. First, the plasmid DNA encoding the luciferase was amplified, extracted, and then purified. Then, the plasmid was linearized, and then purified by chromatographic purification and or LiCl/ethanol precipitation. Finally, the mRNA was enzymatically synthesized in vitro using the linearized plasmid DNA as a template, followed by purification and storage at −80 °C for use.

Example 2 Preparation of tLCNP

tLCNP were synthesized by rapid mixing of ethanol phase and aqueous phase with volume ratio of 1:3 at total flow rate of 12 mL/min using the typical microfluidic device. The aqueous phase is a 50 mM citrate buffer (pH 5.0) containing mRNA or other cargo molecules. The ethanol phase comprises an ionizable lipid (Lipid #4, Immorna), DOTMA (SHOCHEM), cholesterol (Jiangsu Southeast Nanomaterials)/Sito 1,2-Diastearoyl-sn-glycero-3-phosphocholine (DSPC) (Jiangsu Southeast Nanomaterials)/DOPE (AVT)/DOPC (AVT), and 1,2-dimyristoyl-rac-glycero-3-methoxypolyethylene glycol-2000 (DMG-PEG2000) (SINOPEG).

These lipid components were mixed at a molar ratio of 16.7:33.4:38.5:6.6:3.3:1.5 (ionizable lipid:cationic lipid:cholesterol:DSPC:DOPC:DMG-PEG2000). The N/P ratio was fixed as 6. The generated “mRNA@tLCNP” composition was step-wisely purified by TFF system in citrate buffer (pH 5.0) and Tris-HCl buffer (pH 7.2) containing 7.5% sucrose. The mRNA@tLCNP was then diluted to the target concentration, and stored at 2-8 °C for short-term storage (less than 3 months) or −80 °C for long-term storage (over 3 months).

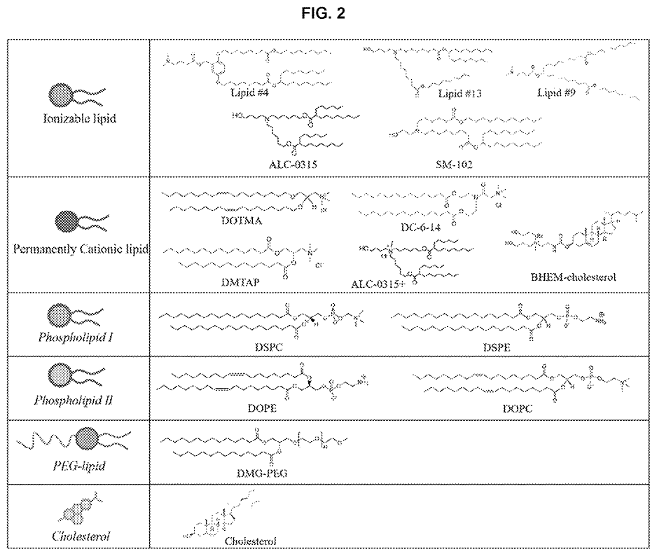

Thus, as shown in FIG. 2, the subject mRNA@tLCNP was synthesized with an ionizable lipid (CI), a permanently cationic lipid (C II), two types of the phospholipids (P I and P II), a stealth lipid DMG-PEG, and one type of sterol (cholesterol, β-sitosterol, or other cholesterol derivative) by microfluidic mixing device.

### Example 3 Characterization of tLCNP

Nanoparticle size and polydispersity index (PDI) of the mRNA@tLCNP prepared in Example 2 above were determined by Dynamic Light Scattering (DLS, Brookhaven 90 Plus PALS), and the mRNA concentration were determined by Ribogreen® assay.

TFF purification process was then used to remove ethanol and to transfer mRNA@tLCNP into Tris-HCl buffer containing sucrose suitable for intravenous administration. Finally, the as-prepared product was characterized by DLS and Ribogreen® assay to determine its size and encapsulation efficiency (EE)/concentration.

### Example 4 In Vivo Evaluation of the tLCNP

The series of in vivo experiments described herein demonstrate the flexibility and optimal conditions for lung-targeting and mRNA expression using the subject tLCNP delivery system.

All animal experiments were approved by the Institutional Animal Care and Use Committee of the Hangzhou Normal University and were consistent with governmental regulations of China for the care and use of animals. Briefly, BALB/c mice (female, 6-8 weeks of age) were placed into groups of n=3. The mice were intravenously injected with luciferase mRNA@tLCNP (dose of mRNA: 5 μg) dispersed in Tris-HCl buffer (pH 7.2) containing 7.5% sucrose. Mice were injected with D-luciferin potassium (150 mg kg-1, intraperitoneal injection) and imaged using an IVIS Lumina system (Perkin Elmer)₆ hours after injection. The organs were then isolated and imaged with the same method.

New Zealand white rabbit, (female, 15 weeks of age) was also used to evaluate lung-targeting. Specifically, a dose of 100 μg mRNA was intravenously injected at the ear margin, IVIS Lumina imaging was performed at 6 hours after injection and RBD determining was performed 4 hours post-injection.

Doping DOTMA into Lipid #4

Luciferase mRNA@tLCNP samples with Lipid #4/DOTMA ratio of 1:0, 3:1, 1:1, 1:2, 1:3, and 1:4 (i.e., Lipid #4 percentage of 100%, 75%, 50%, 33%, 25%, and 20% of the total charged lipid (Lipid #4+DOTMA), respectively, were prepared by the protocol as described above in Example 2. This series of samples covered a range of DOTMA proportion (in relation to total Lipid #4/DOTMA) from about 0-80% (i.e., 0%, 25%, 50%, 67%, 75%, and 80% cationic lipid DOTMA of the total charged lipid (Lipid #4+DOTMA), respectively).

TABLE 1
Synthesis conditions and characterizations, including size and encapsulation efficiency
(EE %), of tLCNP samples prepared with different Lipid #4/DOTMA ratio.

| | Synthesis conditions | | | | | | |
|---|---|---|---|---|---|---|---|
| Lipid | Lipid #4:DOTMA:DSPC:Chol:DMG- | N/P | DLS | | DLS-TFF | | EE |
| #4:DOTMA | PEG | Ratio | Size(nm) | PDI | Size(nm) | PDI | % |
| 1:0 | 50:0:10:38.5:1.5 | 6 | 121.72 | 0.244 | 109.58 | 0.221 | 97.5 |
| 3:1 | 37.5:12.5:10:38.5:1.5 | 6 | 86.23 | 0.211 | 137.22 | 0.184 | 99.6 |
| 1:1 | 25:25:10:38.5:1.5 | 6 | 78.40 | 0.190 | 132.93 | 0.173 | 99.7 |
| 1:2 | 16.7:33.4:10:38.5:1.5 | 6 | 89.48 | 0.232 | 159.27 | 0.202 | 99.9 |
| 1:3 | 12.5:37.5:10:38.5:1.5 | 6 | 82.07 | 0.217 | 148.46 | 0.167 | 99.9 |
| 1:4 | 10:40:10:38.5:1.5 | 6 | 86.73 | 0.187 | 155.43 | 0.168 | 99.9 |

As shown in Table 1, all the mRNA@tLCNP samples had a small size (less than 200 nm) suitable for intended therapeutic use, and had PDI less than 0.25, suggesting good uniformity in size distribution. They also showed excellent encapsulation efficiency (EE %) of over 97% (over 99.5% with the presence of DOTMA). Notably, all LNPs contains different proportions of Lipid #4/DOTMA mixture have particle sizes under 100 nm based on the DLS measurement, most less than 90 nm in size, with one less than 80 nm. See Table 1.

TABLE 2
Data summarized the percentage of luciferase expression in major
organs: liver, spleen, and lung, counted from the expression of
major organs: liver, stomach, kidney, spleen, lung, and heart.

| Expression Percentage | Liver | Spleen | Lung |
|---|---|---|---|
| Lipid #4:DOTMA = 1:0 | 93.5% | 5.0% | 0.5% |
| Lipid #4:DOTMA = 3:1 | 26.3% | 38.3% | 34.7% |
| Lipid #4:DOTMA = 1:1 | 26.3% | 14.2% | 58.2% |
| Lipid #4:DOTMA = 1:2 | 11.2% | 8.5% | 78.8% |
| Lipid #4:DOTMA = 1:3 | 22.1% | 8.1% | 68.3% |
| Lipid #4:DOTMA = 1:4 | 11.1% | 5.9% | 81.0% |

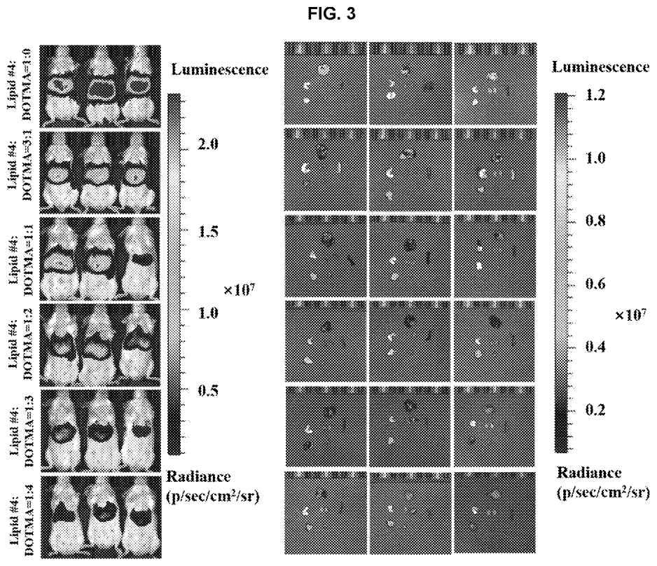

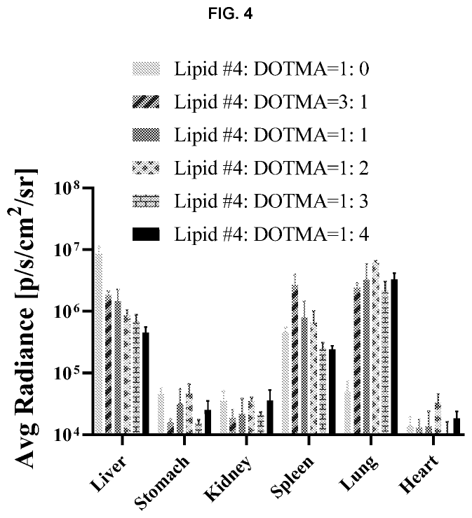

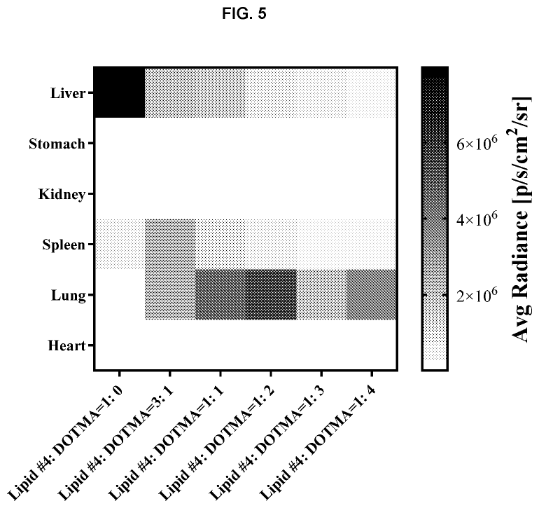

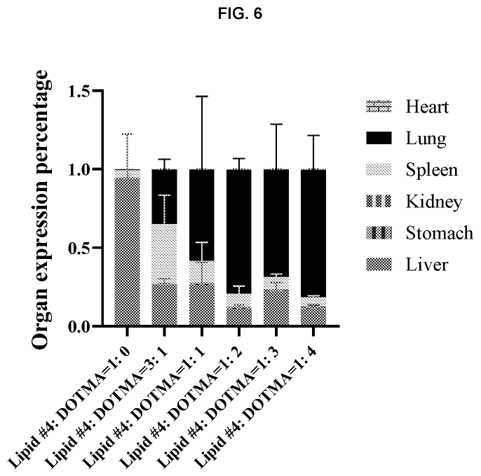

As shown in FIGS. 3-6 and Table 2, with the doping percentage of DOTMA in Lipid #4 LNP increased from 0% to 80%, the organ-targeted expression of mRNA expression gradually moved from liver to spleen, then to lung with targeting efficiency of 81.0%.

Considering the almost comparable lung-targeting efficiency between 78.8% of Lipid #4/DOTMA=1:2 (i.e., 33% Lipid #4/67% DOTMA) and 81.0% of Lipid #4/DOTMA=4:1 (i.e., 80% Lipid #4/20% DOTMA), the Lipid #4/DOTMA=1:2 (i.e., 33% Lipid #4/67% DOTMA) is a potential optimal to balance safety and efficiency.

Doping DOTMA into Other Different Ionizable Lipids Based LNP

To demonstrate that lung-targeting can be achieved with other ionizable lipid-based tLCNP system, three other types of ionizable lipids, including MC3, Lipid #9, and Lipid #13, were used to replace Lipid #4 to formulate tLCNP.

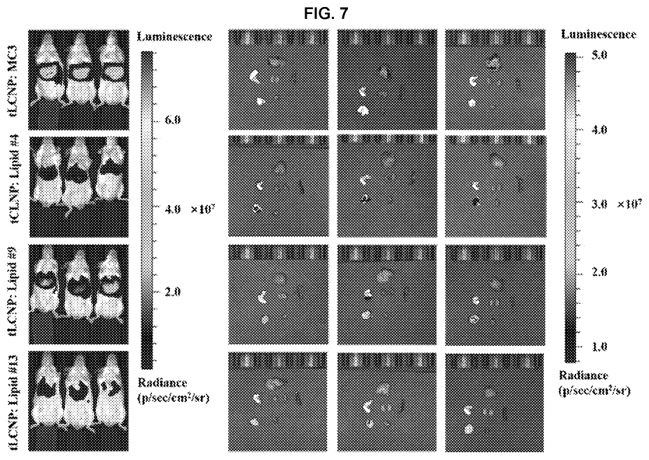

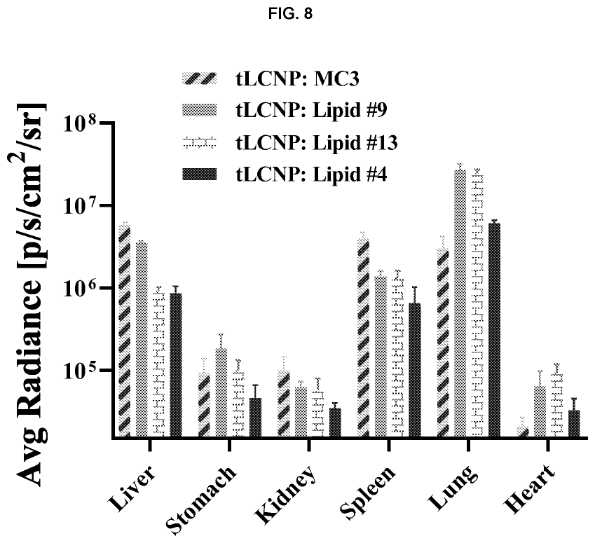

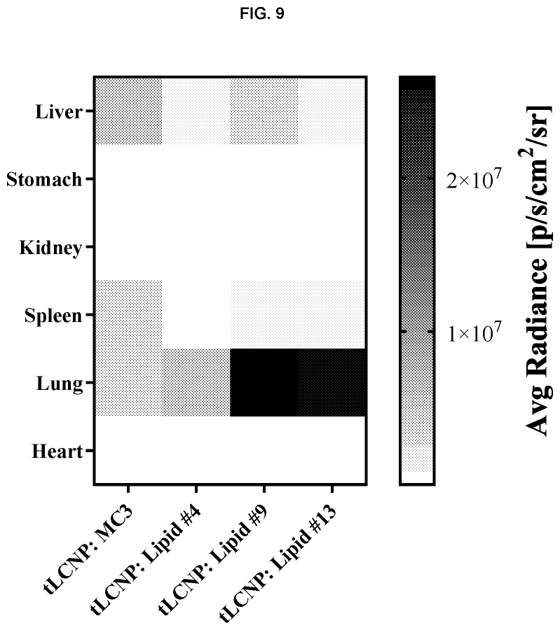

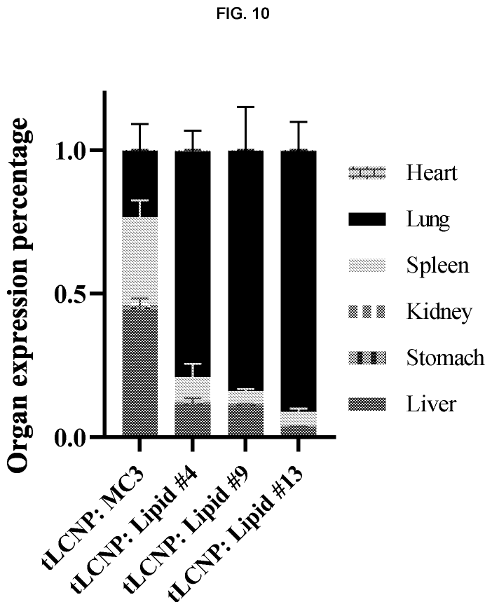

As shown in FIGS. 7-10 and Tables 3-4, although the sizes of the Lipid #9 tLCNP and the Lipid #13 tLCNP were over 200 nm, the larger sizes apparently had no significant influence on lung-targeting, in that both of these tLCNPs achieved a high lung-targeting efficiency to 83.8% and 90.8%, respectively (Table 4).

TABLE 3
Synthesis conditions and characterizations, including size and encapsulation
efficiency (EE %), of tLCNP samples prepared with different ionizable lipids.

| | Synthesis conditions | | | | | | |
|---|---|---|---|---|---|---|---|
| | Ionizable | | | | | | |
| | Lipid:DOTMA:DSPC:Chol:DMG- | N/P | DLS | | DLS-TFF | | EE |
| Ionizable lipid | PEG | Ratio | Size(nm) | PDI | Size(nm) | PDI | % |
| MC3 | 16.7:33.4:10:38.5:1.5 | 6 | 116.92 | 0.076 | 145.85 | 0.081 | 99.9 |
| Lipid #4 | 16.7:33.4:10:38.5:1.5 | 6 | 89.48 | 0.232 | 159.27 | 0.202 | 99.9 |
| Lipid #9 | 16.7:33.4:10:38.5:1.5 | 6 | 159.39 | 0.101 | 256.96 | 0.099 | 99.7 |
| Lipid #13 | 16.7:33.4:10:38.5:1.5 | 6 | 131.54 | 0.174 | 243.89 | 0.090 | 99.8 |

TABLE 4
Data summarized the percentage of luciferase expression in major
organs: liver, spleen, and lung, counted from the expression of
major organs: liver, stomach, kidney, spleen, lung, and heart.

| Expression Percentage | Liver | Spleen | Lung |
|---|---|---|---|
| tLCNP:MC3 | 44.6% | 30.5% | 23.3% |
| tLCNP:Lipid #4 | 11.2% | 8.5% | 78.8% |
| tLCNP:Lipid #9 | 11.0% | 4.3% | 83.8% |
| tLCNP:Lipid #13 | 3.4% | 4.9% | 90.8% |

However, MC3 tLCNP has no lung-targeting capability. While not wishing to be bound by any particular theory, this discrepancy may be caused by the presence of ester bond on the branch tail of the ionizable lipid #4, ionizable lipid #9 and ionizable lipid #13. In other words, ester bond located in hydrophobic tail of ionizable lipid seems to have significant impact on lung-targeting. The data presented herein also shows that a series of ionizable lipids can be used in the lung-targeting tLCNP of the invention (Table 4).

### Regulating the Total Percentage of Lipid #4 and DOTMA Lipids

Partly in view of the smaller size of tLCNP prepared based on Lipid #4 that facilitates the quality control for next development, the following screening and optimization was carried out based on tLCNP of Lipid #4.

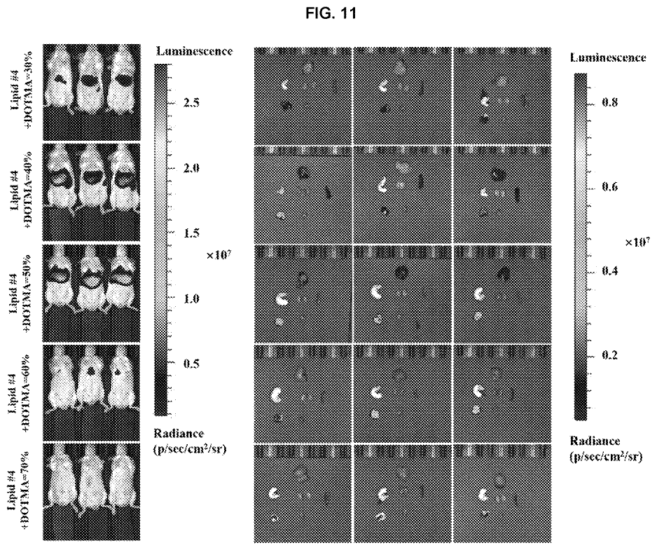

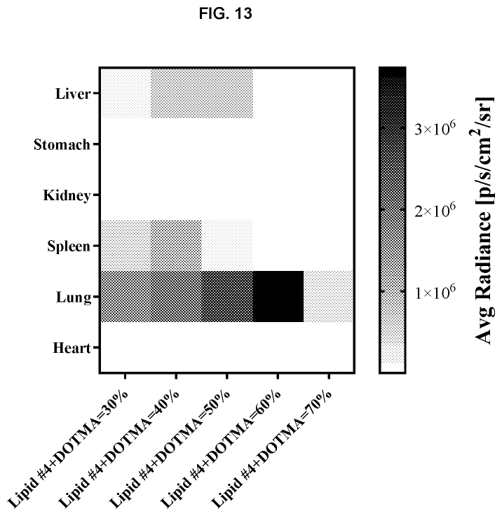

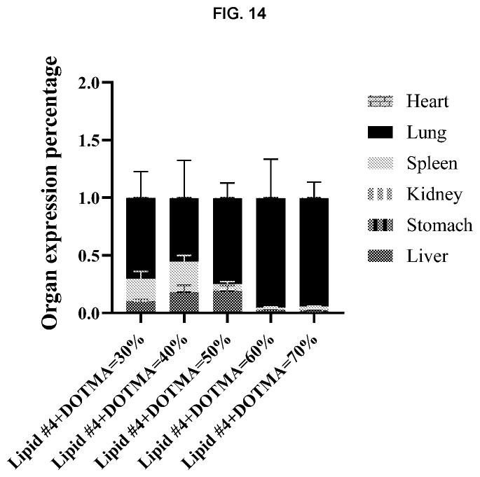

To further optimize lung-targeting and expression efficiency, different percentage of total charged lipids (Lipid #4+DOTMA) were used to investigate the effect of the total charged lipid percentage. As shown in Tables 5-6 and FIGS. 11-14, the lung-targeting efficiency has a total charged lipid percentage-dependent increasing tendency, and the highest lung-targeting is achieved with 60% of total charged lipids (Lipid #4+DOTMA), whereas further elevating the total charged lipid percentage to 70% did not contribute to higher lung-targeting.

TABLE 5
Synthesis conditions and characterizations, including size and encapsulation
efficiency (EE %), of tLCNP samples prepared with different percentage
of total Lipid #4 + DOTMA with 30%, 40%, 50%, 60%, and 70%.

| | Synthesis conditions | | | | | | |
|---|---|---|---|---|---|---|---|
| Lipid | Lipid | | | | | | |
| #4 + DOTMA | #4:DOTMA:DSPC:Chol:DMG- | N/P | DLS | | DLS-TFF | | EE |
| percentage | PEG | Ratio | Size(nm)0 | PDI | Size(nm) | PDI | % |
| 30% | 10:20:14:53.9:2.1 | 6 | 77.38 | 0.161 | 109.57 | 0.123 | 99.7 |
| 40% | 13.3:26.6:12:46.2:1.8 | 6 | 77.14 | 0.156 | 128.44 | 0.143 | 99.8 |
| 50% | 16.7:33.4:10:38.5:1.5 | 6 | 73.85 | 0.228 | 137.31 | 0.191 | 99.1 |
| 60% | 20:40:8:30.8:1.2 | 6 | 78.53 | 0.176 | 126.94 | 0.161 | 99.7 |
| 70% | 23.3:46.6:6:23.1:0.9 | 6 | 64.15 | 0.258 | 97.92 | 0.173 | 99.5 |

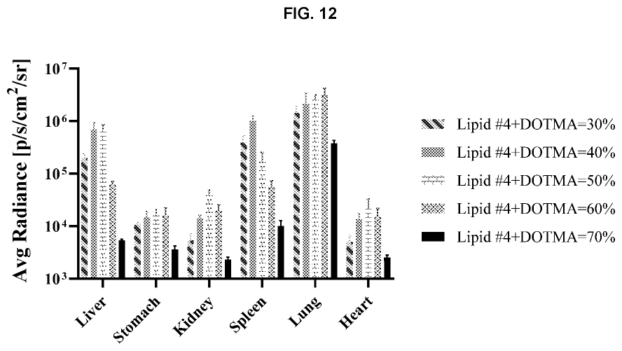

Moreover, expression intensity in lung had a similar tendency, within 30%-60% total charged lipid percentage, however, 70% total charged lipid percentage leads to a dramatically decrease of expression (FIG. 12 and Table 6).

TABLE 6
Data summarized the percentage of luciferase expression in major
organs: liver, spleen, and lung, counted from the expression of
major organs: liver, stomach, kidney, spleen, lung, and heart.

| Expression Percentage | Liver | Spleen | Lung |
|---|---|---|---|
| Lipid #4 + DOTMA = 30% | 9.7% | 19.1% | 70.2% |
| Lipid #4 + DOTMA = 40% | 17.5% | 26.2% | 55.2% |
| Lipid #4 + DOTMA = 50% | 18.0% | 5.0% | 74.4% |
| Lipid #4 + DOTMA = 60% | 2.0% | 2.0% | 94.9% |
| Lipid #4 + DOTMA = 70% | 1.0% | 3.0% | 94.0% |

### Regulating the N/P Ratio

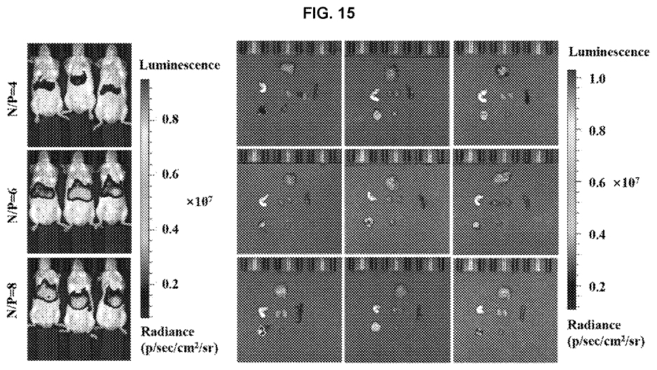

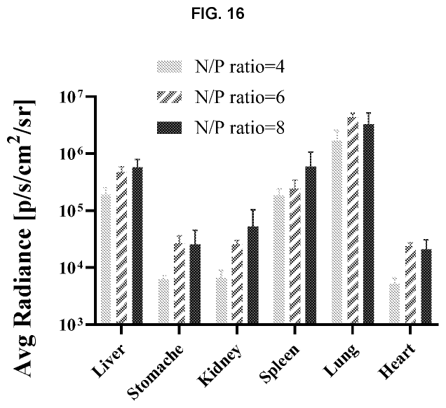

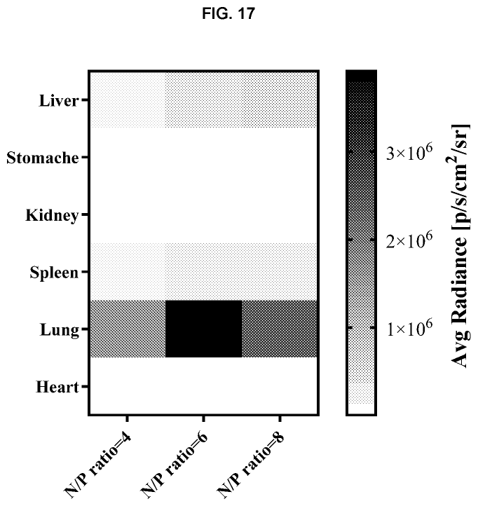

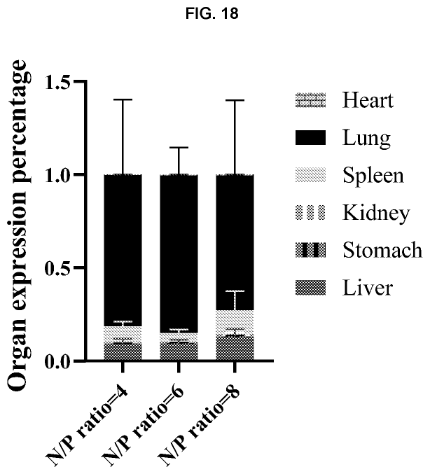

N/P ratio was also optimized in this tLCNP delivery system as shown in FIGS. 15-18 and Tables 7-8. N/P=6 was found to be the optimal compared to the other two tested values, regarding targeting efficiency and expression intensity in lung (Table 8).

TABLE 7
Synthesis conditions and characterizations, including size and encapsulation efficiency
(EE %), of tLCNP samples prepared with different N/P ratio of 4, 6, and 8.

| | Synthesis conditions | | | | | |
|---|---|---|---|---|---|---|
| | Lipid | | | | | |
| N/P | #4:DOTMA:DSPC:Chol:DMG- | DLS | | DLS-TFF | | EE |
| ratio | PEG | Size(nm) | PDI | Size(nm) | PDI | % |
| 4 | 16.7:33.4:10:38.5:1.5 | 87.69 | 0.241 | 136.54 | 0.195 | 99.6 |
| 6 | 16.7:33.4:10:38.5:1.5 | 77.66 | 0.243 | 145.55 | 0.175 | 99.6 |
| 8 | 16.7:33.4:10:38.5:1.5 | 89.80 | 0.214 | 152.33 | 0.184 | 98.7 |

TABLE 8
Data summarized the percentage of luciferase expression in major
organs: liver, spleen, and lung, counted from the expression of
major organs: liver, stomach, kidney, spleen, lung, and heart.

| Expression Percentage | Liver | Spleen | Lung |
|---|---|---|---|
| N/P ratio = 4 | 9.0% | 9.0% | 81.2% |
| N/P ratio = 6 | 9.0% | 5.0% | 84.6% |
| N/P ratio = 8 | 13.0% | 13.0% | 72.4% |

### Boosting Targeting and Expression Efficiency by Doping Another Phospholipid

The above data demonstrated that the subject tLCNP (such as certain Lipid #4 based tLCNP) can achieve over 90% lung-targeting. However, the use of a high percentage of charged lipid in some of these formulations may lead to potentially higher side effects.

TABLE 9
Synthesis conditions and characterizations, including size and encapsulation
efficiency (EE %), of tLCNP samples prepared with different DSPC/DOPE ratio.

| | Synthesis conditions | | | | | | |
|---|---|---|---|---|---|---|---|
| | Lipid | | | | | | |
| | #4:DOTMA:DSPC:DOPE:Chol:DMG- | N/P | DLS | | DLS-TFF | | EE |
| DSPC:DOPE | PEG | ratio | Size(nm) | PDI | Size(nm) | PDI | % |
| 1:0 | 16.7:33.4:10:0:38.5:1.5 | 6 | 90.88 | 0.115 | 115.08 | 0.106 | 99.2 |
| 1:1 | 16.7:33.4:5:5:38.5:1.5 | 6 | 73.55 | 0.064 | 97.77 | 0.147 | 99.0 |
| 0:1 | 16.7:33.4:0:10:38.5:1.5 | 6 | 60.71 | 0.071 | 97.20 | 0.181 | 99.1 |

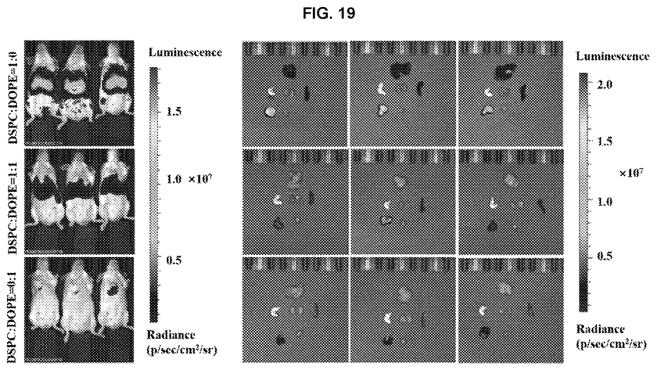

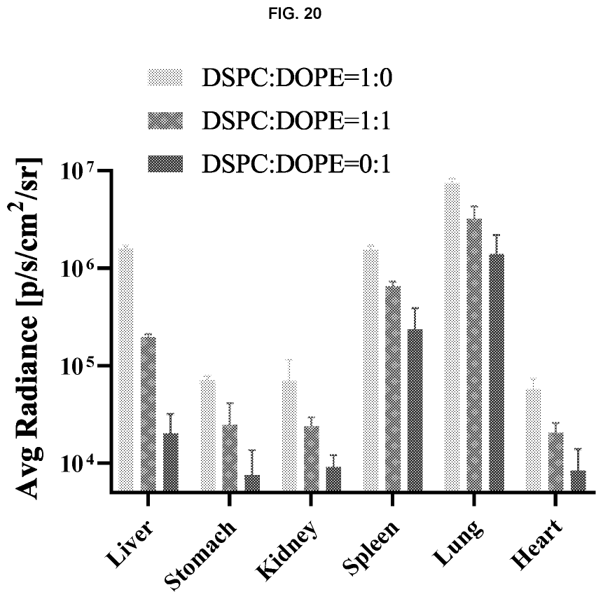

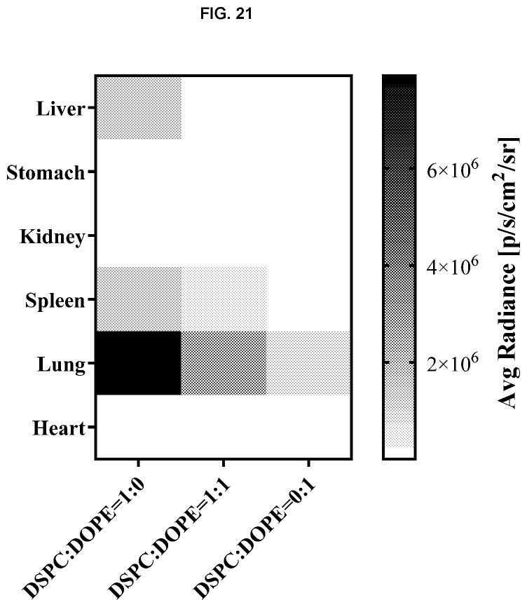

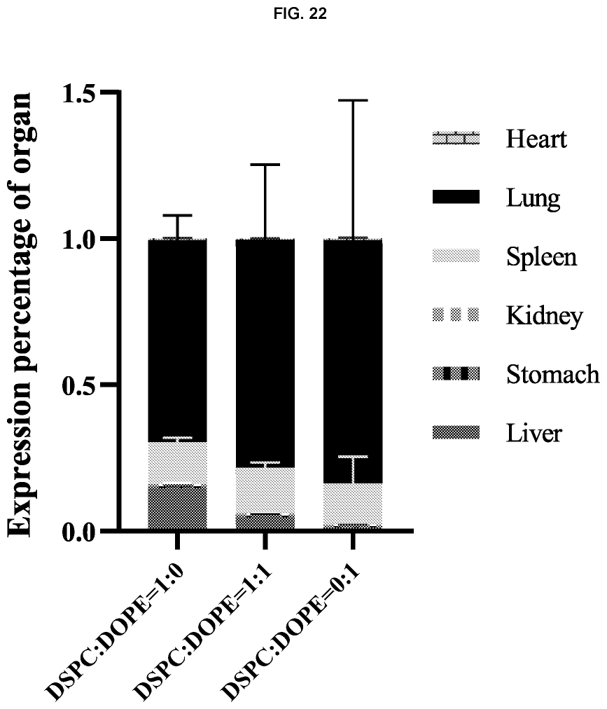

Data presented herein (FIGS. 19-22 & Tables 9-10) provides a further optimization to increase overall lung-targeting and expression, based on a moderate total charged lipid percentage of about 50%. Under this embodiment, DSPC was partially or completely replaced with DOPE (e.g., doping DOPE to DSPC) to alter delivery from the liver to the lungs, thus enabling lung-targeting efficiency to be enhanced to about 83.2% (FIG. 22 & Table 10), yet the mRNA expression is somewhat declined (FIGS. 20 & 21).

TABLE 10
Data summarized the percentage of luciferase expression in major
organs: liver, spleen, and lung, counted from the expression of
major organs: liver, stomach, kidney, spleen, lung, and heart.

| Expression Percentage | Liver | Spleen | Lung |
|---|---|---|---|
| DSPC:DOPE = 1:0 | 14.7% | 14.5% | 69.0% |
| DSPC:DOPE = 1:1 | 4.7% | 15.7% | 77.9% |
| DSPC:DOPE = 0:1 | 1.2% | 14.1% | 83.2% |

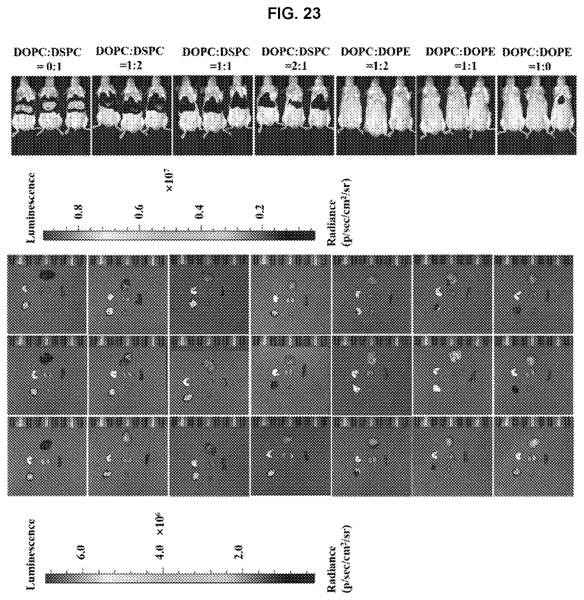

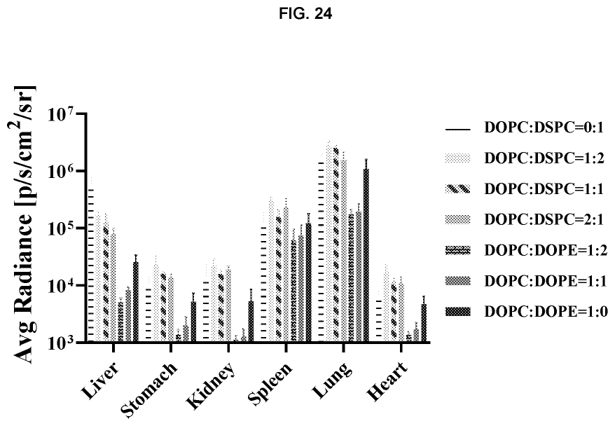

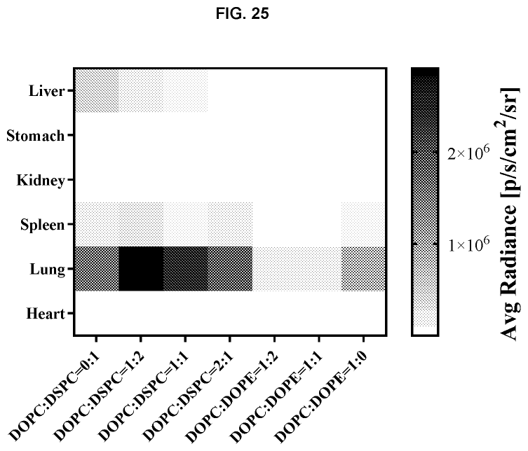

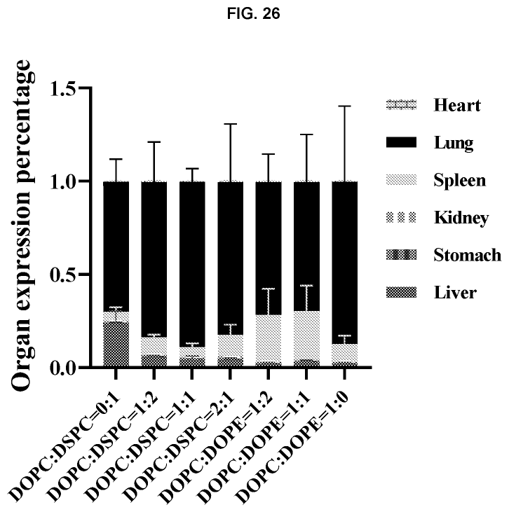

Further combination optimizations were tested, between DSPC and DOPC, or DOPE and DOPC (FIGS. 23-26 and Tables 11-12). The data showed that the expression of the marker gene luciferase in all groups was also predominantly located in the lungs. In particular, on calculating the relative expression in each organ, doping DOPC into DSPC at a ratio of 1:2 and 1:1 efficiently altered delivery from the liver and spleen to the lungs, thus enabling lung-targeting to be elevated from 69.8% to 83.3% and 88.6%. Meanwhile, expression level was also elevated from 1.757×10⁶ p/s/cm²/sr to 2.627×10⁶ p/s/cm²/sr and 2.530×10⁶ p/s/cm²/sr, respectively (Table 12).

TABLE 11
Synthesis conditions and characterizations, including size and encapsulation efficiency
(EE %), of tLCNP samples prepared with different DOPC/DSPC ratio or DOPC/DOPE ratio.

| | Synthesis conditions | | | | | | |
|---|---|---|---|---|---|---|---|
| | Lipid | | | | | | |
| DOPC | #4:DOTMA:DOPC:DSPC:Chol:DMG- | N/P | DLS | | DLS-TFF | | EE |
| doping | PEG | Ratio | Size(nm) | PDI | Size(nm) | PDI | % |
| DOPC:DSPC | | | | | | | |
| 0:1 | 16.7:33.4:0:10:38.5:1.5 | 6 | 70.32 | 0.238 | 130.55 | 0.209 | 99.6 |
| 1:2 | 16.7:33.4:3.3:6.6:38.5:1.5 | 6 | 64.64 | 0.180 | 123.59 | 0.187 | 99.6 |
| 1:1 | 16.7:33.4:5:5:38.5:1.5 | 6 | 59.02 | 0.169 | 121.89 | 0.189 | 99.3 |
| 2:1 | 16.7:33.4:6.6:3.3:38.5:1.5 | 6 | 61.05 | 0.191 | 119.09 | 0.196 | 99.6 |
| DOPC:DOPE | | | | | | | |
| 1:2 | 16.7:33.4:3.3:6.6:38.5:1.5 | 6 | 62.61 | 0.196 | 115.40 | 0.218 | 99.5 |
| 1:1 | 16.7:33.4:5:5:38.5:1.5 | 6 | 59.68 | 0.221 | 120.51 | 0.225 | 99.6 |
| 1:0 | 16.7:33.4:10:0:38.5:1.5 | 6 | 65.61 | 0.214 | 119.47 | 0.207 | 99.6 |

TABLE 12
Data summarized the percentage of luciferase expression in major
organs: liver, spleen, and lung, counted from the expression of
major organs: liver, stomach, kidney, spleen, lung, and heart.

| Expression | | | Lung | |
|---|---|---|---|---|
| Percentage/Intensity | Liver | Spleen | Percentage | Intensity |
| DOPC:DSPC = 0:1 | 23.7% | 5.2% | 69.8% | 1.757 × 10⁶ |
| DOPC:DSPC = 1:2 | 5.4% | 9.3% | 83.3% | 2.627 × 10⁶ |
| DOPC:DSPC = 1:1 | 4.4% | 5.5% | 88.6% | 2.530 × 10⁶ |
| DOPC:DSPC = 2:1 | 4.1% | 11.8% | 81.8% | 1.559 × 10⁶ |
| DOPC:DOPE = 1:2 | 2.1% | 25.1% | 71.3% | 1.737 × 10⁵ |
| DOPC:DOPE = 1:1 | 2.9% | 26.2% | 69.1% | 1.940 × 10⁵ |
| DOPC:DOPE = 1:0 | 2.1% | 9.8% | 86.9% | 1.081 × 10⁶ |

The above data showed that introducing primary amine-capped phospholipid with unsaturated fat chains, such as DOPE, can strengthen lung targeting, without facilitating endosomal escape and subsequent expression (FIGS. 19-22 & Table 10). Interestingly, introducing quaternary ammonium-capped phospholipid with unsaturated fat chains, such as DOPC, generated a dual beneficial effect regarding lung-targeting and expression intensity (FIGS. 23-26 & Table 12).

Overall, these results demonstrated that the two approaches described herein-doping permanently cationic lipid (C₂ lipid) and quaternary ammonium-capped phospholipid with unsaturated fat chains (PII lipid) into traditional LNP synergistically enhanced both lung-targeting and cargo mRNA expression efficiency.

### Example 5 Comparison of Different Lung-Targeted Delivery Systems

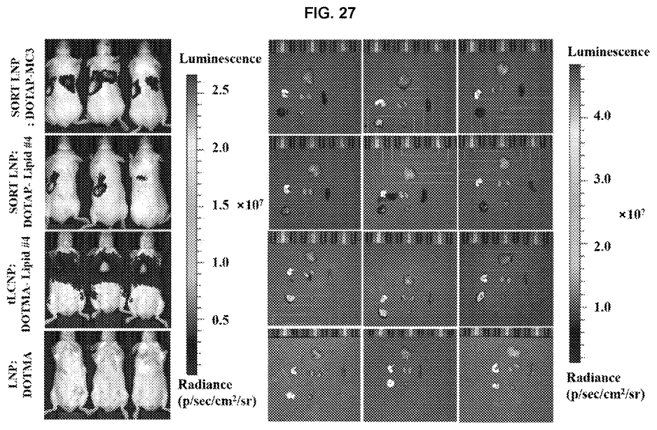

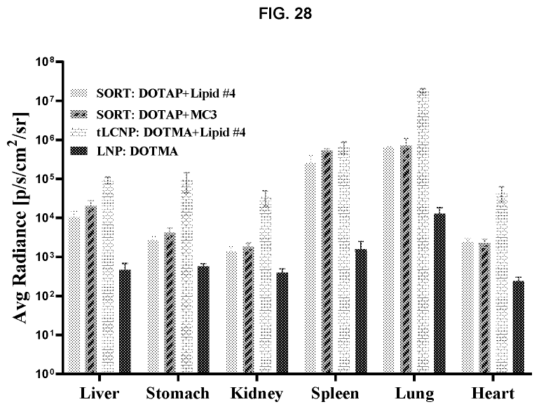

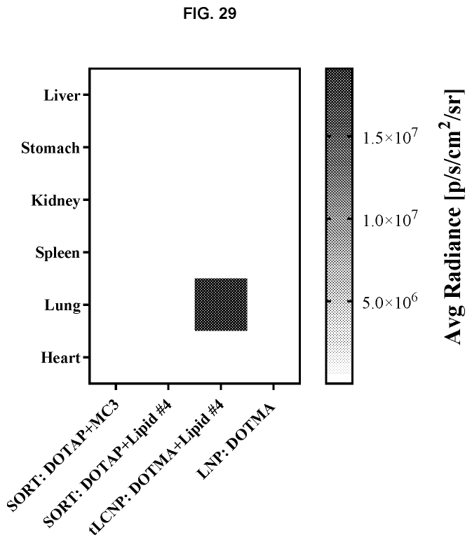

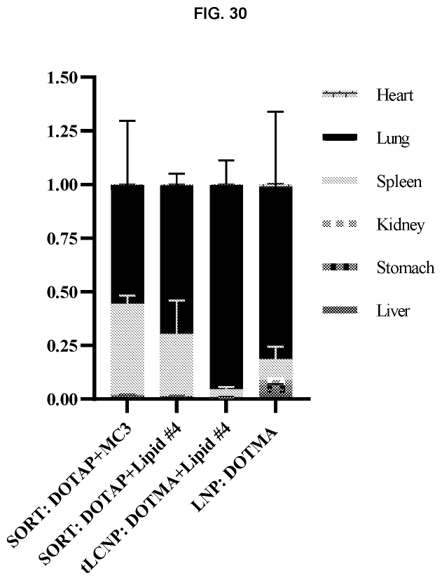

To validate the superiority of the subject tLCNP based lung-targeting delivery system, a head-to-head comparison between SORT LNP and the subject tLCNP regarding lung-targeting delivery efficiency was performed in in vivo experiments (FIGS. 27-30 & Tables 13-14).

TABLE 13
Sample information, including synthesis conditions and characterizations: size and encapsulation efficiency (EE %).

| Different | Synthesis conditions | | | | | | |
|---|---|---|---|---|---|---|---|
| lung-targeted | | Lipid/mRNA | DLS | | DLS-TFF | | EE |
| delivery system | Lipid Ratio | (N/P ratio) | Size(nm) | PDI | Size(nm) | PDI | % |
| SORT LNP:DOTAP + | DOTAP:MC3:DSPC:Chol:DMG-PEG = | 20 | 151.86 | 0.150 | 179.84 | 0.114 | 99.3 |
| MC3 | 50:25:5:19.25:0.75 | | | | | | |
| SORT LNP:DOTAP + | DOTAP:Lipid #4:DSPC:Chol:DMG-PEG = | 20 | 171.86 | 0.155 | 202.42 | 0.119 | 99.4 |
| Lipid #4 | 50:25:5:19.25:0.75 | | | | | | |
| tLCNP:DOTMA + | DOTMA:Lipid #4:DSPC:DOPC:Chol:DMF-PEG = | 6 | 64.21 | 0.113 | 77.86 | 0.232 | 98.5 |
| Lipid #4 | 33.4:16.7:6.6:3.3:38.5:1.5 | | | | | | |
| LNP:DOTMA | DOTAP:DSPC:Chol:DMG-PEG = | 6 | 106.39 | 0.230 | 123.7 | 0.146 | 98.7 |
| | 50:10:38.5:1.5 | | | | | | |

The subject tLCNP based on DOTMA and Lipid #4 was prepared at an optimized composition and lipid ratio formulation: Lipid #4:DOTMA:DSPC:DOPC:Chol:DMG-PEG=16.7:33.4:6.6:3.3:38.5:1.5. Two SORT LNP formulations containing DOTAP+MC3 and DOTAP+Lipid #4, respectively, were prepared using the same procedure (Table 13).

The sizes and CEs of all products were in a controlled range for application. Similarly, the marker gene luciferase was predominantly expressed in the lungs in all groups. Although the SORT LNP containing DOTAP and Lipid #4 showed a higher lung-targeting efficiency (69.3%) over that (55.4%) of the SORT LNP containing DOTAP and MC3 (reported formulation), both of which were significantly lower than that (95.2%) of the subject tLCNP (Table 14).

Furthermore, tLCNP exhibited an overwhelming expression intensity of luciferase (1.83×10⁷ p/s/cm²/sr) compared to the expression intensity of luciferase via the delivery formulation both of SORT LNP:DOTAP+Lipid #4 (7.09×10⁵) and SORT LNP:DOTAP+MC3 (6.26×10⁵ p/s/cm²/sr) (Table 14).

TABLE 14
Data summarized the percentage of luciferase expression
in major organs: liver, spleen, and lung.

| Expression | | | Lung | |
|---|---|---|---|---|
| Percentage/Intensity | Liver | Spleen | Percentage | Intensity |
| SORT LNP:DOTAP + MC3 | 1.6% | 42.3% | 55.4% | 7.093 × 10⁵ |
| SORT LNP:DOTAP + | 1.1% | 28.8% | 69.3% | 6.260 × 10⁵ |
| Lipid #4 | | | | |
| tLCNP:DOTMA + Lipid #4 | 0.5% | 3.4% | 95.2% | 1.830 × 10⁷ |
| LNP:DOTMA | 2.9% | 9.9% | 79.8% | 1.277 × 10⁴ |

The above results demonstrated that the subject tLCNP showed a distinct advantage over SORT LNP, regarding selective delivery of nucleic acid therapeutic agent to lung, as well as expression efficiency, showing a great potential in clinical translations.

Finally, DOTMA based LNP was also prepared to have a comparison with tLCNP regarding lung-targeting. The data shows that, even though DOTMA-based LNP (without Lipid #4) also had a high lung-targeting efficiency (79.8%), its expression intensity (1.27×10⁴ p/s/cm²/sr) was far less than that of the subject tLCNP system.

The evidence presented above indicates that the subject tLCNP delivery system has a great potential in clinical application to treat or prevent specific lung diseases.

### Example 6 Evaluating Lung-Targeting of tLCNP on Larger Animals

To show that the subject lung-targeting delivery system is not depended on particular animal species, a test was performed in rabbits by injection of the subject tLCNP loaded with luciferase-encoding mRNA, and mRNA expressing receptor binding domain (RBD) sequence of the SARS-COV-2 virus (“COVID-19 RBD”), as shown in Table 15. Luciferase expression was determined by IVIS Lumina imaging in vivo and ex vivo. RBD expression in major organs (liver, lung, and spleen) was determined by ELISA.

TABLE 15
Sample information of tLCNP loading luciferase
mRNA and COVID RBD mRNA, respectively.

| mRNA | Synthesis | DLS | | DLS-TFF | | EE |
|---|---|---|---|---|---|---|
| Cargo | conditions | Size(nm) | PDI | Size(nm) | PDI | % |
| Luciferase | tLCNP, N/P = 6 | 63.00 | 0.177 | 81.95 | 0.202 | 99.9% |
| COVID-19 RBD | tLCNP, N/P = 6 | 61.87 | 0.198 | 77.13 | 0.165 | 100% |

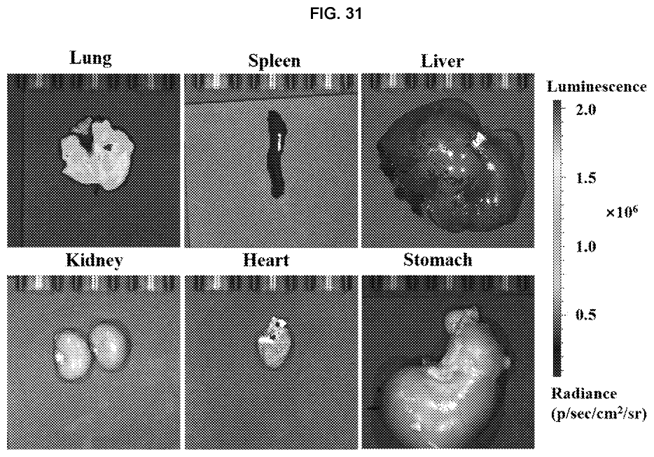

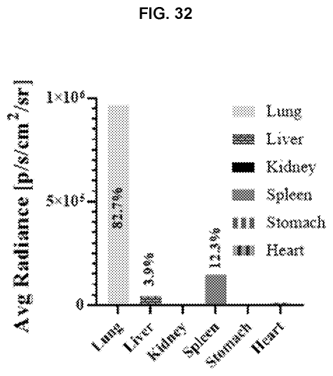

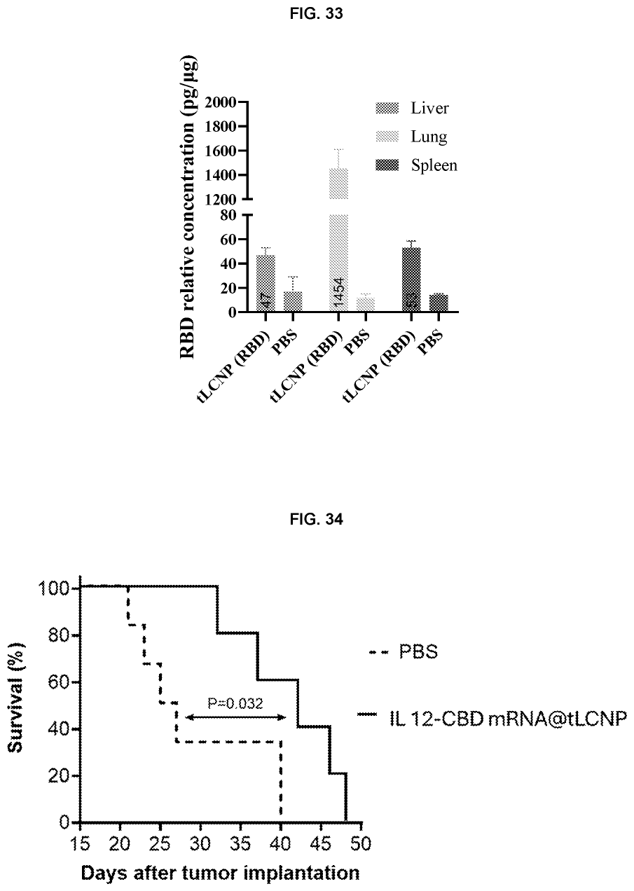

The data indicated that lung-targeting in rabbit had a similar tendency-reaching to about 82.7% (FIGS. 31 and 32). Moreover, ELISA test determining RBD concentration was performed on liver, lung, and spleen of rabbits at 4 hours post-injection of tLCNP (RBD) and PBS. As shown in FIG. 33, the detected relative RBD concentration in lung (1454 μg/μg) was overwhelmingly higher than that in liver (47 μg/μg) and that in spleen (53 μg/μg).

These data demonstrated that the lung-targeting tLCNP delivery system of the invention does not depend on particular animal species or particular cargo molecule/therapeutic agent for its observed advantageous characteristics.

### Example 7 Evaluating Targeted Therapy of IL12-CBD mRNA@tLCNP on Mice with Metastatic LL2 Lung Tumors

This example demonstrates the delivery efficiency of the subject tLCNP for a loaded mRNA drug, for use in treating cancer.

Specifically, mRNA encoding mouse interleukin 12-collagen binding domain fusion protein (IL12-CBD) was synthesized, for delivery (using the subject tLCNP) into and treatment for developed primary and metastatic LL2 lung cancer models.

Briefly, IL12-CBD mRNA@tLCNP was prepared using methods essentially as described in Example 2, and was subsequently characterized essentially as described in Example 3. The size/PDI and encapsulation efficiency were found to be 74.67 nm/0.135 and 94.5%, respectively.

Mouse Lewis lung carcinoma (LLC) cells were purchased from ATCC (Manassas, VA, USA) and cultured in RPMI DMEM containing FBS (10%), penicillin (50 μg/mL) and streptomycin (50 μg/mL). Cells were maintained at 37 °C in a humidified atmosphere with 5% CO₂. Then, 100 μl of LLC cell (2× 10⁵ per mouse) suspension was injected per mouse into the mouse tail vein (Six-to eight-week-old C₅₇BL/6 male mice) to generate the lung tumor model. Mice were randomly divided into the IL12-CBD mRNA@tLCNP (n=10) treatment group, and the PBS control group (n=10). At days 9, 13, 17, and 21 after tumor cell injection, mice were treated with intravenous administration of IL12-CBD mRNA@tLCNP (10 μg IL12-CBD mRNA/mouse) or PBS. Mortality was measured every day.

As shown in FIG. 34, IL12-CBD mRNA@tLCNP treated lung tumor mice exhibited an increased survival rate compared to that of the PBS treated control group.

1. A targeted tLCNP that preferentially delivers a therapeutic agent encompassed/formulated within the tLCNP to the lungs of a mammal, the tLCNP comprises:
(1) a first lipid composition comprising an ionizable lipid C1 and a cationic lipid C2;
(2) a second lipid composition comprising a first phospholipid PI and a second phospholipid PII;
(3) a sterol lipid; and,
(4) a stealth lipid;
wherein:
(A) the ionizable lipid C1 has the formula of C₁-a, C₁-b, C₁-c or C₁-d: 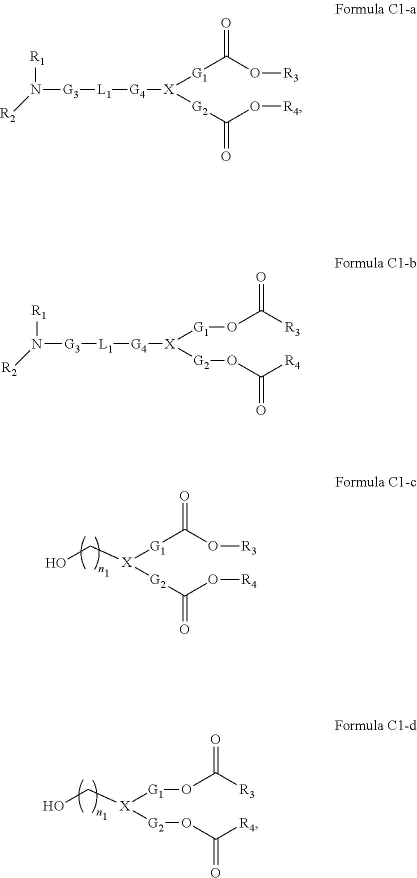
wherein:
R₁ and R₂ are each independently —CH₃, —(CH₂)₁₋₈—CH₃, or —(CH₂)₁₋₈—OH;
G₁ and G₂ are independently —(CH₂)₁₋₁₀—;
G₃ and G₄ are independently —(CH₂)₀₋₅—;
L₁ is —O—(C═O)—O—, —O—(C═O)—, —(C═O)—O—, —S—S—, —O—;
R₃ and R₄ are each independently 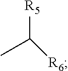
R₅ is —H, or —(CH₂)₇CH₃, —(CH═CH)(CH₂)₅CH₃, C₁₋₁₄ alkyl, or C₁₋₁₄ alkenyl;
R₆ is —(CH₂)₇CH₃, —(CH═CH)(CH₂)₅CH₃, C₁₋₂₀ alkyl, or C₁₋₂₀ alkenyl;
X is C or 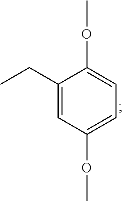
and,
n₁ is an integer of 2-8;
(B) the cationic lipid C2 has the formula of C₂-a, C₂-b, C₂-c or C₂-d: 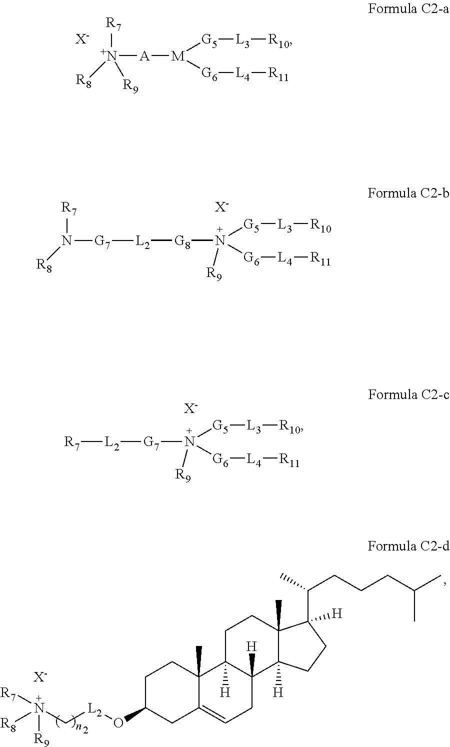
wherein:
R₇, R₈, and R₉ are each independently —CH₃, —(CH₂)₁₋₈—CH₃, or —(CH₂)₁₋₈—OH;
R₁₀ and R₁₁ are each independently 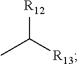
R₁₂ is —H, —(CH₂)₇CH₃, —(CH═CH)(CH₂)₅CH₃, C₁₋₁₄ alkyl, or C₁₋₁₄ alkenyl;
R₁₃ is —(CH₂)₇CH₃, —(CH═CH)(CH₂)₅CH₃, C₁₋₂₀ alkyl, or C₁₋₂₀ alkenyl;
M is carbon or nitrogen;
A is —CH₂—, —G₇-L₂-G₈- or —(CH₂)₁₋₅—(C═O)—, wherein-(C═O)— is linked to M;
G₅, G₆, G₇ and G₈ are independently —(CH₂)₀₋₁₀—;
n₂ is an integer of 0-10;
L₂, L₃, and L₄ are each independently —CH₂—, —O—(C═O)—O—, —O—(C═O)—, —(C═O)—O—, —S—S—, —O—, —S—, —HN—(C═O)—;
X⁻ is monovalent anion (e.g., a halo group), such as Cl and Br “;
(C) optionally, the first phospholipid PI has the formula of PI: 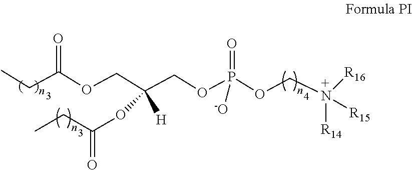
wherein R₁₄, R₁₅, and R₁₆ are each independently —H, —CH₃, —(CH₂)₁₋₆—CH₃, or —(CH₂)₁₋₆—OH;
n₃ in each instance is independently an integer of 5-20; and,
n₄ is an integer of 1-5;
(D) optionally, the second phospholipid PII has the formula of PII: 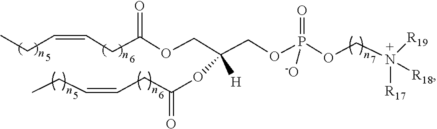
wherein R₁₇, R₁₈, and R₁₉ are each independently —H, —CH₃, —(CH₂)₁₋₆—CH₃, or —(CH₂)₁₋₆—OH;
n₅ and n₆, in each instance, is independently an integer of 5-8;
n₇ is an integer of 1-5; and
(E) optionally, wherein the steroid comprises, consists essentially of, or consists of cholesterol.

2. (canceled)

3. The tLCNP of claim 1, wherein the stealth lipid comprises, consists essentially of, or consists of a PEGylated-lipid having a PEG (—O—CH₂—CH₂—) repeat, such as DMG-PEG2000

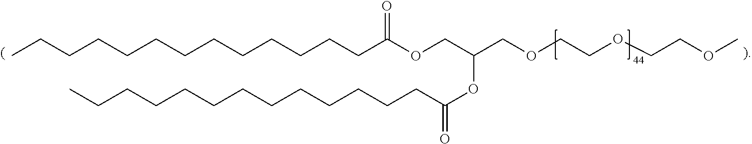

4. The tLCNP of claim 1, wherein R₅ is H, —(CH₂)₃CH₃, —(CH₂)₅CH₃, —(CH₂)₇CH₃, or —(CH₂)₉CH₃; or
wherein R₅ comprises H, —(CH═CH)(CH₂)₃CH₃, —(CH₂)₃ (CH═CH) CH₂CH₃, —(CH═CH)(CH₂)₄CH₃, —(CH═CH)(CH₂)₅CH₃, or —(CH═CH)(CH₂)₇CH₃.

5. (canceled)

6. The tLCNP of claim 1, wherein R₆ comprises-(CH₂)₃CH₃, —(CH₂)₅CH₃, —(CH₂)₇CH₃, or —(CH₂)₉CH₃;
wherein R₆ comprises-(CH═CH)(CH₂)₃CH₃, —(CH₂)₃ (CH═CH) CH₂CH₃, —(CH═CH)(CH₂)₄CH₃, —(CH═CH)(CH₂)₅CH₃, or —(CH═CH)(CH₂)₇CH₃;
wherein R₆ comprises H, —(CH₂)₃CH₃, —(CH₂)₅CH₃, —(CH₂)₇CH₃, —(CH₂)₉CH₃, or —(CH₂)₁₁CH₃, optionally, wherein the cationic lipid C2 is DOTMA, and the ionizable lipid C1 is Lipid #4; or
wherein R₆ comprises H, —(CH═CH)(CH₂)₃CH₃, —(CH₂)₃ (CH═CH) CH₂CH₃, —(CH═CH)(CH₂)₄CH₃, —(CH═CH)(CH₂)₅CH₃, —(CH═CH)(CH₂)₇CH₃, or —(CH₂)₇ (CH═CH)(CH₂)₇CH₃.

7-9. (canceled)

10. The tLCNP of claim 1, wherein R₇ comprises-(CH₂)₃CH₃, —(CH₂)₅CH₃, —(CH₂)₇CH₃, —(CH₂)₉CH₃, or —(CH₂)₁₁CH₃; or
wherein R₇ comprises-(CH═CH)(CH₂)₃CH₃, —(CH₂)₃ (CH═CH) CH₂CH₃, —(CH═CH)(CH₂)₄CH₃, —(CH═CH)(CH₂)₅CH₃, —(CH═CH)(CH₂)₇CH₃, or —(CH₂) z (CH═CH)(CH₂)₇CH₃.

11. (canceled)

12. The tLCNP of claim 1, wherein said ionizable lipid C1 comprises

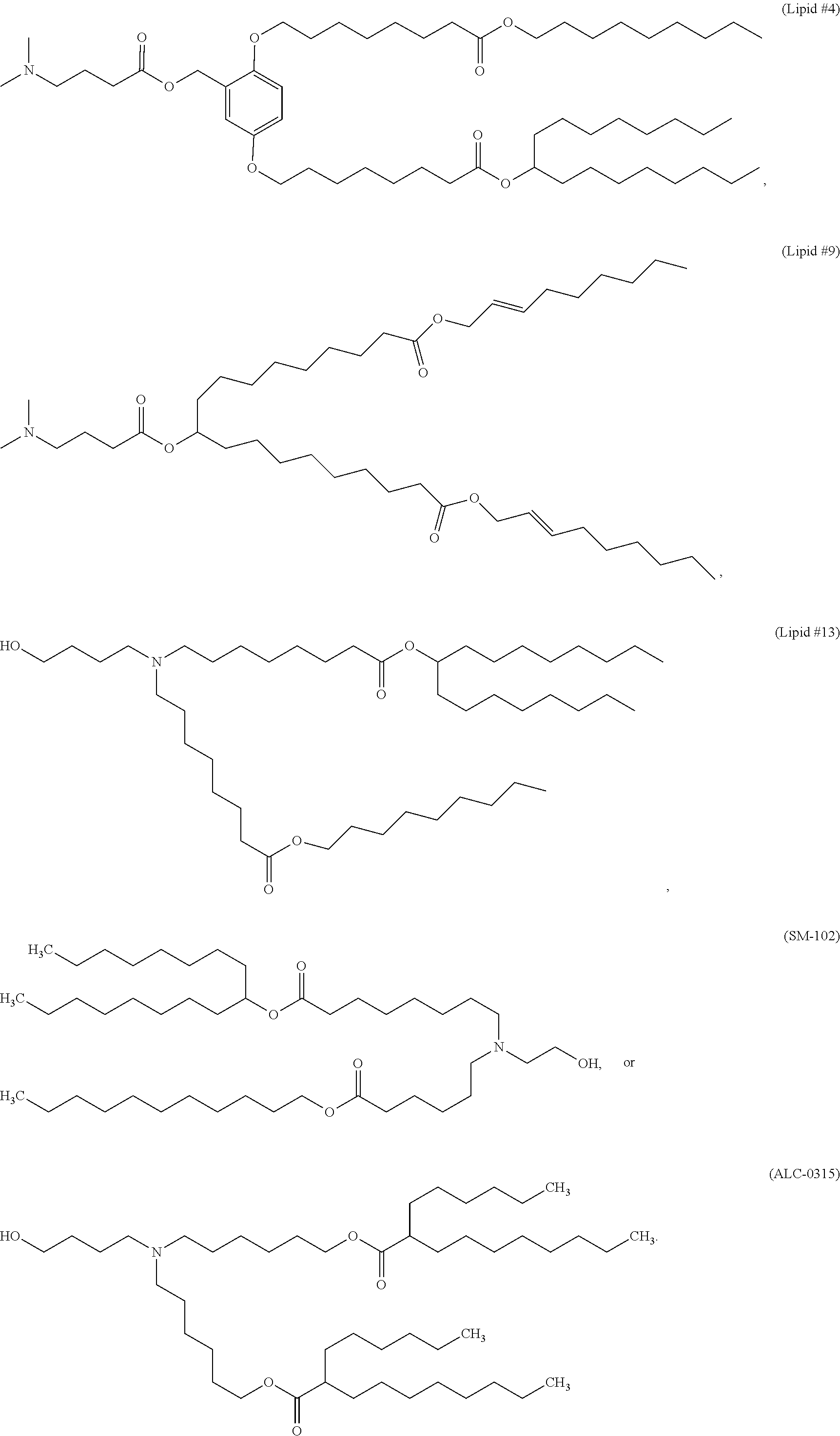

13. The tLCNP of claim 1, wherein said cationic lipid C2 comprises

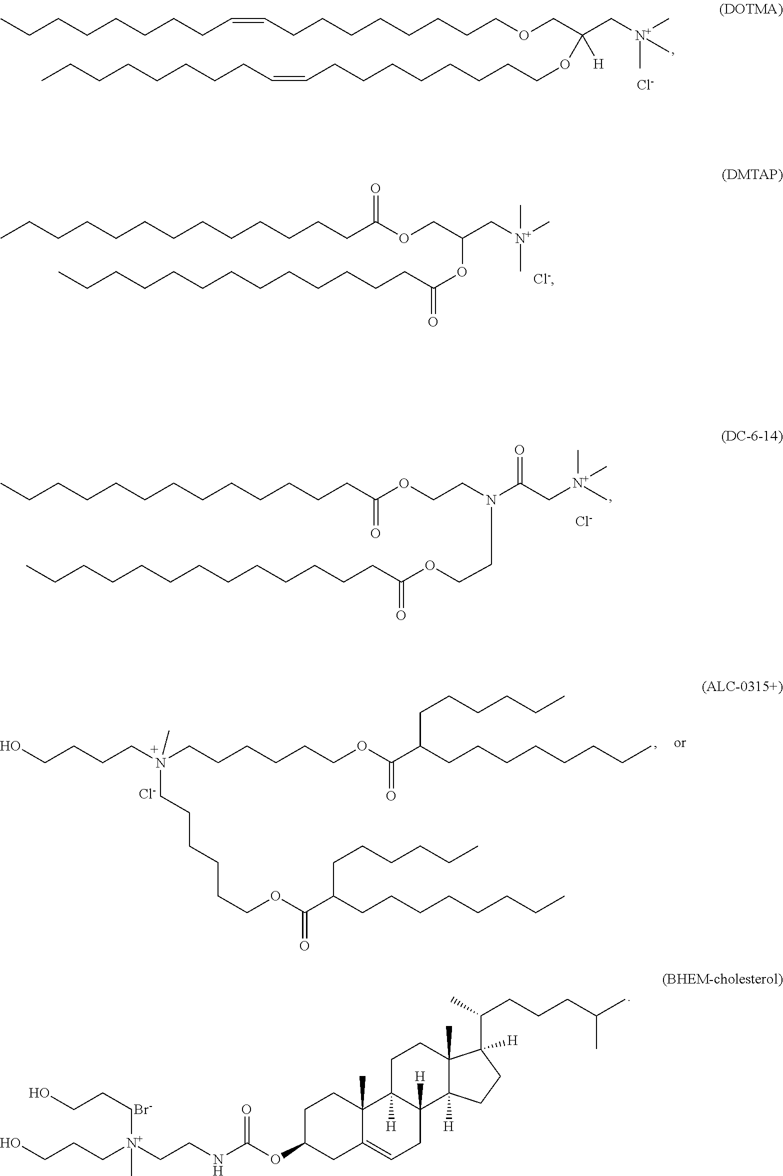

14. The tLCNP of claim 1, wherein said first phospholipid PI comprises

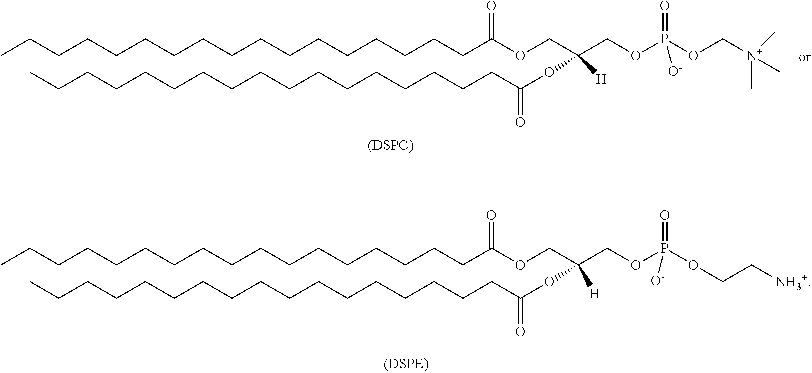

15. The tLCNPt of claim 1, wherein said second phospholipid PII comprises

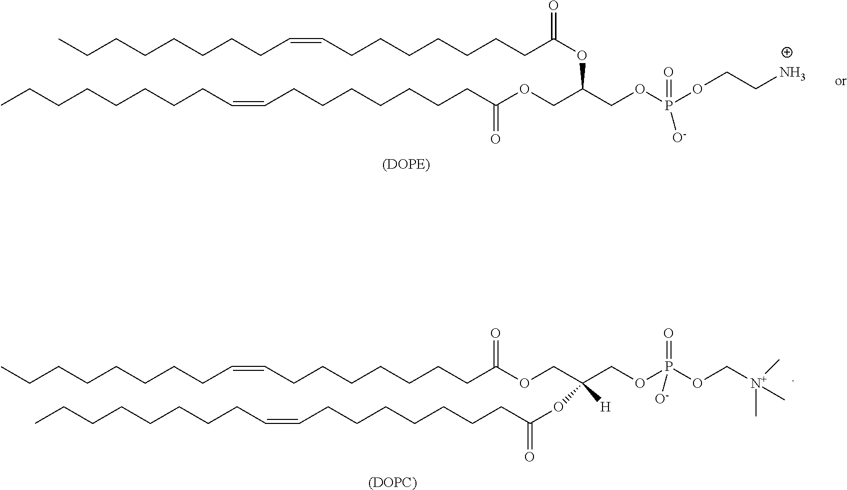 [SMILES: CCCCCCCC/C=C\CCCCCCCC(=O)OC[C@H](COP(=O)([O-])OCC[NH3+])OC(=O)CCCCCCC/C=C\CCCCCCCC ; CCCCCCCC/C=C\CCCCCCCC(=O)OC[C@H](COP(=O)([O-])OC[N+](C)(C)C)OC(=O)CCCCCCC/C=C\CCCCCCCC ; "(DOPC)"]

16. The tLCNP of claim 1, wherein the molar ratio of the cationic lipid C2 over the ionizable lipid C1 is about 1:3 to about 4:1, about 1:1 to about 4:1, about 2:1 to about 4:1, about 3:1 to about 4:1, about 1:1, about 2:1, about 3:1, or about 4:1.

17. (canceled)

18. The tLCNP of claim 1, wherein the molar percentage of (1) (or the first lipid composition) over (1)-(4) is about 30-70%, about 40-60%, about 50-60%, about 30%, about 40%, about 50%, about 60%, or about 70%,
optionally, wherein the molar ratio of the cationic lipid C2 (such as DOTMA) over the ionizable lipid C1 (such as Lipid #4) is about 2:1.

19. (canceled)

20. The tLCNP of claim 1, comprising:
(a) about 30%-70%, about 40%-60%, or about 50% of the first lipid composition in molar percentage;
(b) about 5%-30% (such as about 5-15%, or about 10%) of the second lipid composition in molar percentage, optionally, the molar ratio of the first phospholipid PI (such as DSPC) over the second phospholipid PII (such as DOPC) is from 1:0 (100% DSPC) to 1:2 (33% DSPC), such as 2:1 (67% DSPC) or 1:1 (50% DSPC);
(c) about 10%-40% (such as about 35-40%, or about 38.5%) of the steroid (such as cholesterol) in molar percentage;
(d) about 0%-5% (such as about 2-3%, or about 1.5%) of the stealth lipid (such as DMG-PEG2000) in molar percentage; or
(e) a combination of any one of the above, wherein the total molar percentage of the combination is 100%.

21. The tLCNP of claim 1, wherein the therapeutic agent comprises a polynucleotide (including DNA and RNA, such as mRNA, siRNA, miRNA, ASO, and pDNA), a hydrophobic and/or hydrophilic small molecule compound (e.g., with a molecular weight of under 1000 Da, 750 Da, 500 Da, 250 Da, or 100 Da), or a bioactive lipid (such as fatty acid, or monoglyceride),
optionally, wherein the therapeutic agent comprises a polynucleotide, such as an RNA (e.g., an siRNA, a miRNA, a pri-miRNA, a messenger RNA (mRNA), a clustered regularly interspaced short palindromic repeats (CRISPR) related nucleic acid, a single guide RNA (sgRNA), a CRISPR-RNA (crRNA), a trans-activating crRNA (tracrRNA), a plasmid DNA (pDNA), a transfer RNA (tRNA), an antisense oligonucleotide (ASO), a guide RNA, a double stranded DNA (dsDNA), a single stranded DNA (ssDNA), a single stranded RNA (ssRNA), and a double stranded RNA (dsRNA)); and
optionally, wherein the tLCNP is injected into the mammal intravenously;
further optionally, wherein the RNA is a self-replicating mRNA, a non-self-replicating mRNA, or a circular RNA; and/or wherein the N/P molar ratio (first lipid composition (permanent cationic lipid+ionizable lipid) lipid Nitrogen: polynucleotide Phosphate) of thetLCNP is about 4, 6, or 8, such as 6.

22-24. (canceled)

25. The tLCNP of claim 1, wherein at least about 60%, 70%, 75%, 80%, 85%, 90%, or 95% of the therapeutic agent is delivered to the lungs when the tLCNP is administered to the mammal;
wherein no more than about 30%, 25%, 20%, 15%, 10%, or 5% of the therapeutic agent is delivered to the liver (or other non-lung tissues combined) when the tLCNP is administered to the mammal; and/or
wherein about 1-15%, about 2-10%, or about 3-5% of the therapeutic agent is delivered to the spleen when the tLCNP is administered to the mammal.

26. (canceled)

27. The tLCNP of claim 1, which on average has a size less than 200 nm, less than 150 nm, less than 100 nm, less than 90 nm, or less than 80 nm.

28. The tLCNP of claim 1, which has a polydispersity index (PDI) of between about 0.10-0.25, or between about 0.15-0.20.

29. (canceled)

30. A pharmaceutical composition comprising the tLCNP of claim 1; and a pharmaceutically acceptable carrier or excipient.

31. A method of delivering a therapeutic agent selectively or preferentially to the lungs of a mammal, comprising administering to the mammal the therapeutic agent encompassed or formulated in the tLCNP of claim 1 to the mammal;
optionally, wherein the mammal is human;
optionally, wherein the tLCNP is injected into the mammal intravenously; and
optionally, wherein the therapeutic agent comprises a polynucleotide, such as an RNA (e.g. mRNA).

32-36. (canceled)

37. A method of preparing the tLCNP of claim 1, the method comprising mixing a first organic liquid phase comprising the first lipid composition, the second lipid composition, the steroid, and the stealth lipid of any one of claims 1-21, in an organic solvent (e.g., ethanol) that is miscible with water; and a second aqueous phase comprising the therapeutic agent of any one of claims 1-29, in order to form the tLCNP;
optionally, wherein the method further comprises step-wisely purification of the tLCNP by a Tangential Flow Filtration (TFF) system;
optionally, wherein said organic solvent in the first organic liquid phase comprises, consists essentially of, or consists of ethanol;
optionally, wherein the first organic liquid phase and the second aqueous phase are mixed in a microfluidic device, optionally with a volume ratio of 1:3, and/or optionally at a total flow rate of about 10-15 mL/min. (e.g., about 12 mL/min.);
optionally, wherein the aqueous phase comprises a 50 mM citrate buffer (pH 5.0); and
optionally, wherein the step-wisely purification is performed in the TFF system in a citrate buffer (pH 5.0) and Tris-HCl buffer (pH 7.2) containing 7.5% sucrose.

38-42. (canceled)
